(19) United States
(12) Patent Application Publication
(10) Pub. No.: US 2016/0272980 A1
(43) Pub. Date: Sep. 22, 2016

(54) METHOD FOR TARGETED MODIFICATION OF ALGAE GENOMES

(71) Applicant: TOTAL MARKETING SERVICES, Puteaux (FR)

(72) Inventors: Philippe DUCHATEAU, Draveil (FR); Fayza DABOUSSI, Chelles (FR)

(21) Appl. No.: 14/442,323

(22) PCT Filed: Nov. 18, 2013
(86) PCT No.: PCT/IB2013/002974

§ 371 Date: May 12, 2015

Related U.S. Application Data
(60) Provisional application No. 61/839,165, filed on Jun. 25, 2013.
(60) Provisional application No. 61/727,444, filed on Nov. 16, 2012.

(51) Int. Cl. C12N 15/82; C12N 15/52; C07K 14/195; C12N 9/22; C12N 9/12; C12R 1/89

(52) U.S. Cl. CPC C12N 15/8213; C12N 9/1241; C12R 1/89; C07K 14/195; C12N 9/22; C12N 15/52; C12Y 301/00; C12Y 301/11002; C12Y 207/07009; C07K 2319/80

(57) ABSTRACT

The invention relates to a method for modifying genetic material in algal cells that includes the use of rare-cutting endonuclease to target specific genomic sequences. In particular, the invention relates to a method for modifying genetic material in algal cells wherein rare-cutting endonuclease, especially a homing endonuclease or a TALE-Nuclease, is expressed over several generations to efficiently modify said target genome sequences.

The invention relates to a method for modifying genetic material in algal cells that includes the use of rare-cutting endonuclease to target specific sequence. In particular, the invention relates to a method for modifying genetic material in algal cells wherein rare-cutting endonuclease, especially a homing endonuclease or a TALE-Nuclease, is expressed over several generations to efficiently modify said target sequence.

### BACKGROUND OF THE INVENTION

Although algae have been used as a food source by humans for centuries, the significance of their biotechnological interest, especially of microalgae, appeared only in recent decades. Applications of algal products range from simple biomass production for food, feed and fuels to valuable products such as cosmetics, pharmaceuticals, pigments, sugar polymers and food supplements.

Several algal species such as Dunaliella bardawil, Haematococcus pluvialis and Chlorella vulgaris have already been exploited extensively in the past for biotechnological purposes, especially as feed, as a source of pigments like β-carotene or astaxanthin or as food supplements (Steinbrenner and Sandmann 2006; Mogedas, Casal et al. 2009). Most of these organisms are green algae that belonging to a group more related to land plants than other algal groups (Palmer, Soltis et al. 2004). Chromophytic algae on the other hand only recently moved into the forefront and their biochemistry and genetics have been studied just in the recent years. They comprise important groups like the brown algae, diatoms, xanthophytes, eustigmatophytes and others, but also the colourless oomycetes (Tyler, Tripathy et al. 2006). Research on chromophytic algae received a strong boost after publication of several genomes including those of the diatoms Thalassiosira pseudonana (Armbrust, Berges et al. 2004) and Phaeodactylum tricornutum (Bowler, Allen et al. 2008).

Diatoms are one of the most ecologically successful unicellular phytoplankton on the planet, being responsible for approximately 20% of global carbon fixation, representing a major participant in the marine food web. There are two major potential commercial or technological applications of diatoms. First, Diatoms are able to accumulate abundant amounts of lipid suitable for conversion to liquid fuels and because of their high potential to produce large quantities of lipids and good growth efficiencies, they are considered as one of the best classes of algae for renewable biofuel production. Second, Diatoms have a cell wall consisting of silica (silica exoskeletons called frustules) with intricated and ornate structures on the nano- to micro-scale. These structures exceed the diversity and the complexity capable by man-made synthetic approaches, and Diatoms are being developed as a source of materials mainly for nanotechnological applications (Lusic, Radonic et al. 2006).

Although the genomes of several algal species have now been sequenced, very few genetic tools to explore microalgal genetics are available at this time, which considerably limits the use of these organisms for various biotechnological applications. The ability to perform targeted genomic manipulations within algal genome was recently facilitated by the use of homing endonuclease (WO 2012/017329). However, due to low transformation rates and the weak expression of transgenes, this approach remains difficult to perform especially, in diatoms, due to their particular silica cell wall comprising two separate valves (or shells). Stable and transient transgene expression systems have been reported in algae—for review see (Hallmann 2007)—as in most organisms, but in most cases, transient expression is sought for the expression of DNA modifying enzymes due to their potential genotoxicity.

As a particular group of microalgae, diatoms are the only major group of eukaryotic phytoplankton with a diplontic life history, in which all vegetative cells are diploid and meiosis produces short-lived, haploid gametes, suggesting an ancestral selection for a life history dominated by a duplicated (diploid) genome. Therefore, in order to create algae, such as diatoms, with new properties, it is deemed necessary to target several alleles or homologous genes concomitantly to cause phenotype effect.

### SUMMARY OF THE INVENTION

Overcoming the above limitations, the inventors have induced bi-allelic or multi-copy knock-out in diatoms by transfection and expression over several generations of transgenes encoding rare-cutting endonucleases, especially engineered endonucleases and TALE-Nucleases. Mosaic clones of such transformed algae cells allowed to isolate a number of descendant cells, where targeted modifications in multi-copy genes or multiple alleles was observed. This new method and its achievements, open the way to the genetic engineering of complex genomes in algae cells.

Thus, the present invention relates to a method for targeted modification of the genetic material of an algal cell using rare-cutting endonucleases, especially by expressing homing endonucleases and TALE-Nuclease over several generations, in particular by stable integration of the transgenes encoding thereof on the chromosome. This method allows inducing targeted insertion (knock-in) or knock-out in several alleles or homologous genes in one experiment run and therefore is facilitating gene stacking. The present invention also encompasses genetically modified algae obtained by this method.

### DESCRIPTION OF THE FIGURES

In addition to the preceding features, the invention further comprises other features which will emerge from the description which follows, as well as to the appended drawings. A more complete appreciation of the invention and many of the attendant advantages thereof will be readily obtained as the same becomes better understood by reference to the following Figures in conjunction with the detailed description below.

FIG. 1: Examples of mutagenic events induced by the PTRI20 meganuclease.

FIG. 2: Mutagenesis induced by PTRI20 meganuclease in the presence of single-chain TREX2 (SCTREX2). A-T7 endonuclease assays on PCR products from the wild type Phaeodactylum tricornutum strain (condition 4) and clones resulting from the transformations with the empty vector (Condition 3), the PTRI20 meganuclease alone (condition 2) and the PTRI20 meganuclease plus SCTREX2 (condition 1 clone A).

FIG. 3: Examples of mutagenic events induced by the PTRI20 meganuclease in the presence of SCTREX2.

FIG. 4: Mutagenesis induced by PTRI02 meganuclease in the presence of single-chain TREX2 (SCTREX2). Characterization of mutagenesis events are characterized by deep sequencing. Genomic DNA of colony lysates from clones derived from the transformation with the PTRI02 meganuclease and SCTREX2 (1-5), and clones resulting from the transformation with the empty vector alone (6-8) was analyzed. A PCR surrounding the PTRI02 specific target was performed and the percentage of mutagenesis frequency induced by the meganuclease in presence of SCTREX2 was determined by deep sequencing analysis of amplicons.

FIG. 5: Examples of mutagenic events induced by the PTRI02 meganuclease in the presence of SCTREX2.

FIG. 6: Frequency of mutagenesis induced by YFP_TALE-Nuclease. Genomic DNA of the clones derived from transformations with TALE-Nuclease or from transformations with the empty vector was extracted. A PCR surrounding the YFP target was performed and the percentage of mutagenesis was determined by a deep sequencing analysis of amplicons centered on the specific target. A sub-clone resulting from clone n° 2 was also analyzed.

FIG. 7: Examples of mutagenic events induced by YFP_TALE-Nuclease.

FIG. 8: Examples of a mutagenic event induced by TP07_TALE-Nuclease

FIG. 9: Example of a mutagenic event induced by TP15_TALE-Nuclease

FIG. 10: Characterization of homologous gene targeting (HGT) events by deep sequencing induced by PTRI02. Genomic DNA of 8 clones transformed with the PTRI02 meganuclease and the DNA matrix (1-8), and clones transformed with DNA matrix and the empty vector (9-10) was analyzed. The percentage of HGT frequency induced by the meganuclease in presence of a DNA matrix was determined by deep sequencing analysis of amplicons.

FIG. 11: Characterization of homologous gene targeting (HGT) events by deep sequencing induced by PTRI20. Genomic DNA of clones transformed with the PTRI20 meganuclease and the DNA matrix (1-3), and clones transformed with DNA matrix and the empty vector (4-5) was analyzed. The percentage of HGT frequency induced by the meganuclease in presence of a DNA matrix was determined by deep sequencing analysis of amplicons.

FIG. 12: Molecular characterization of clones from the transformation of the Phaeodactylum tricornutum (Pt) strain with the TALE-Nuclease targeting the UGPase gene.

FIG. 13: Molecular characterization of clones from the transformation of the Phaeodactylum tricornutum (Pt) strain with the TALE-Nuclease targeting the UGPase gene (experiment 1).

FIG. 14: Molecular characterization of clones from the transformation of the Phaeodactylum tricornutum (Pt) strain with the TALE-Nuclease targeting the UGPase gene (experiment 2).

FIG. 15: Example of a mutagenic event induced by the TALE-Nuclease targeting the UDP glucose pyrophosphorylase gene.

FIG. 16: Phenotypic characterization of Phaeodactylum tricornutum (Pt) strain transformed with the TALE-Nuclease targeting the UGPase gene. Clone 37-7A1: 100% mutated on the UGPase gene, clone 37-3B1 from transformation with the empty vector and the Pt wild type strain were labeled with the lipid probe (Bodipy, Molecular Probe). The fluorescence intensity was measured by flow cytometry. The graphs represent the number of cells function of the fluorescence intensity for 3 independent experiments.

FIG. 17: Mutagenesis induced by the TALE-Nuclease targeting the putative elongase gene. Left panel: PCR realized on clone lysates from the transformations with the empty vector and the putative elongase TALE-Nuclease were performed. Right panel: T7 assay was assessed on 4 clones resulting from the transformation with the putative elongase TALE-Nuclease and on 3 clones resulting from the transformation with the empty vector. The clone 2 is positive for the T7 assay.

FIG. 18: Example of a mutagenic event induced by the TALE-Nuclease targeting the putative elongase gene.

Table 1: Mutagenesis induced by PTRI20 meganuclease.

Table 2: Number of clones obtained after transformation, the number of clones that have integrated the PTRI020 meganuclease and SCTREX2 DNA sequences and the number of clones tested in the T7 assay and Deep sequencing analysis.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention concerns the use of rare-cutting endonucleases to allow efficient targeted genomic engineering of algal cells. In a preferred embodiment, the present invention relates to a method for targeted modification of the genetic material of an algal cell comprising one or several of the following steps:

a) Selecting a nucleic acid target sequence in the genome of an algal cell;

b) Designing a gene encoding a rare-cutting endonuclease to target this sequence;

c) Transfecting said algal cell with vectors comprising said gene encoding said rare-cutting endonuclease to obtain its expression within said cell over several generations;

d) Selecting the cell progeny of said algal cell having a modified target sequence.

Said modified target sequence can result from NHEJ events or homologous recombination. The double strand breaks caused by said rare-cutting endonucleases are commonly repaired through the distinct mechanisms of homologous recombination or non-homologous end joining (NHEJ). Although homologous recombination typically uses the sister chromatid of the damaged DNA as a donor matrix from which to perform perfect repair of the genetic lesion, NHEJ is an imperfect repair process that often results in changes to the DNA sequence at the site of the double strand break. Mechanisms involve rejoining of what remains of the two DNA ends through direct re-ligation (Critchlow and Jackson 1998) or via the so-called microhomology-mediated end joining (Ma, Kim et al. 2003). Repair via non-homologous end joining (NHEJ) often results in small insertions or deletions and can be used for the creation of specific gene knockouts. In one aspect of this embodiment, the present invention relates to a method for targeted modification of the genetic material of an algal cell by expressing rare-cutting endonuclease into algal cell to induce either homologous recombination or NHEJ events.

Said rare-cutting endonuclease according to the present invention refers to any wild type or variant enzyme capable of catalyzing the hydrolysis (cleavage) of bonds between nucleic acids within a DNA or RNA molecule, preferably a DNA molecule. Over the last 15 years, the use of homing endonuclease to successfully induce gene targeting has been well documented starting from straightforward experiments involving wild-type I-Scel to more refined work involving completely re-engineered enzyme (Stoddard, Monnat et al. 2007; Marcaida, Prieto et al. 2008; Galetto, Duchateau et al. 2009; Arnould, Delenda et al. 2011 and WO2011/064736). The endonuclease according to the present invention recognizes and cleaves nucleic acid at specific polynucleotide sequences, further referred to as “nucleic acid target sequence”.

The rare-cutting endonuclease according to the invention can for example be a homing endonuclease also known as meganuclease (Paques and Duchateau 2007). Such homing endonucleases are well-known to the art (see e.g. (Stoddard, Monnat et al. 2007). Homing endonucleases recognize a nucleic acid target sequence and generate a single- or double-strand break.

Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Examples of such endonuclease include I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I or I-Msol.

In a preferred embodiment, the homing endonuclease according to the invention is a LAGLIDADG endonuclease such as I-Scel, I-Crel, I-Ceul, I-Msol, and I-Dmol. In a most preferred embodiment, said LAGLIDADG endonuclease is I-Crel. Wild-type I-Crel is a homodimeric homing endonuclease that is capable of cleaving a 22 to 24 bp double-stranded target sequence.

In the present application, I-Crel variants may be homodimers (meganuclease comprising two identical monomers) or heterodimers (meganuclease comprising two non-identical monomers). It is understood that the scope of the present invention also encompasses the I-Crel variants per se, including heterodimers (WO2006097854), obligate heterodimers (WO2008093249) and single chain meganucleases (WO03078619 and WO2009095793) as non limiting examples, able to cleave one of the sequence targets in the algal genome. The invention also encompasses hybrid variant per se composed of two monomers from different origins (WO03078619).

The invention encompasses both wild-type and variant endonucleases. In a preferred embodiment, the endonuclease according to the invention is a “variant” endonuclease, i.e. an endonuclease that does not naturally exist in nature and that is obtained by genetic engineering or by random mutagenesis. The variant endonuclease according to the invention can for example be obtained by substitution of at least one residue in the amino acid sequence of a wild-type, endonuclease with a different amino acid. Said substitution(s) can for example be introduced by site-directed mutagenesis and/or by random mutagenesis. In the frame of the present invention, such variant endonucleases remain functional, i.e. they retain the capacity of recognizing and specifically cleaving a target sequence. In a more preferred embodiment, nucleic acid encoding the homing endonucleases used in the present invention comprise a part of nucleic acid sequence selected from the group consisting of: SEQ ID NO: 1 and SEQ ID NO: 12.

The variant endonuclease according to the invention cleaves a target sequence that is different from the target sequence of the corresponding wild-type endonuclease. Methods for obtaining such variant endonucleases with novel specificities are well-known in the art.

The present invention is based on the finding that such variant endonucleases with novel specificities can be used to allow efficient targeted modification of the genetic material of an algal cell, thereby considerably increasing the usability of these organisms for various biotechnological applications.

In another preferred embodiment, said rare-cutting endonuclease can be a “TALE-nuclease” (TALE-Nuclease) resulting from the fusion of DNA binding domain derived from a Transcription Activator like Effector (TALE) and one nuclease domain able to cleave a DNA target sequence. TALE-NucleaseS are used to stimulate gene targeting and gene modifications (Boch, Scholze et al. 2009; Moscou and Bogdanove 2009; Christian, Cermak et al. 2010, WO 2011/146121).

Said Transcription Activator like Effector (TALE) corresponds to an engineered TALE comprising a plurality of TALE repeat sequences, each repeat comprising a RVD specific to each nucleotide base of a TALE recognition site. In the present invention, each TALE repeat sequence of said TALE is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. By other amino acid residues is intended any of the twenty natural amino acid residues or unnatural amino acids derivatives.

In another embodiment, said TALE of the present invention comprises between 8 and 30 TALE repeat sequences. More preferably, said TALE of the present invention comprises between 8 and 20 TALE repeat sequences; again more preferably 15 TALE repeat sequences.

In another embodiment, said TALE comprises an additional single truncated TALE repeat sequence made of 20 amino acids located at the C-terminus of said set of TALE repeat sequences, i.e. an additional C-terminal half-TALE repeat sequence. In this case, said TALE of the present invention comprises between 8.5 and 30.5 TALE repeat sequences, “0.5” referring to previously mentioned half-TALE repeat sequence (or terminal RVD, or half-repeat). More preferably, said TALE of the present invention comprises between 8.5 and 20.5 TALE repeat sequences, again more preferably, 15.5 TALE repeat sequences. In a preferred embodiment, said half-TALE repeat sequence is in a TALE context which allows a lack of specificity of said half-TALE repeat sequence toward nucleotides A, C, G, T. In a more preferred embodiment, said half-TALE repeat sequence is absent. In another embodiment, said TALE of the present invention comprises TALE like repeat sequences of different origins. In a preferred embodiment, said TALE comprises TALE like repeat sequences originating from different naturally occurring TAL effectors. In another preferred embodiment, internal structure of some TALE like repeat sequences of the TALE of the present invention are constituted by structures or sequences originated from different naturally occurring TAL effectors. In another embodiment, said TALE of the present invention comprises TALE like repeat sequences. TALE like repeat sequences have a sequence different from naturally occurring TALE repeat sequences but have the same function and/or global structure within said core scaffold of the present invention.

TALE-nuclease have been already described and used to stimulate gene targeting and gene modifications (Christian, Cermak et al. 2010). Such engineered TAL-nucleases are commercially available under the trade name TALEN™ (Cellectis, 8 rue de la Croix Jarry, 75013 Paris, France).

In particular embodiment, said TALE-Nuclease according to the invention targets a sequence within a UDP-glucose pyrophosphorylase or a putative elongase gene, preferably within sequence having at least 70%, more preferably 80%, 85%, 90%, 95% identity with SEQ ID NO: 41 or SEQ ID NO: 52. More preferably, the TALE-nuclease targets a sequence having at least 70%, preferably 75%, 80%, 85%, 90%, 95% with the SEQ ID NO: 44 or 55.

The rare-cutting endonuclease according to the invention can also be for example a chimeric Zinc-Finger nuclease (ZFN) resulting from the fusion of engineered zinc-finger domains with the nuclease catalytic domain of a restriction enzyme such as Fokl (Porteus and Carroll 2005) or a chemical endonuclease (Eisenschmidt, Lanio et al. 2005; Arimondo, Thomas et al. 2006; Simon, Cannata et al. 2008; Cannata, Brunet et al. 2008).

By “nuclease catalytic domain” is intended the protein domain comprising the active site of an endonuclease enzyme. Such nuclease catalytic domain can be, for instance, a “cleavage domain” or a “nickase domain”. By “cleavage domain” is intended a protein domain whose catalytic activity generates a Double Strand Break (DSB) in a DNA target. By “nickase domain” is intended a protein domain whose catalytic activity generates a single strand break in a DNA target sequence.

The catalytic domain is preferably a nuclease domain and more preferably a domain having endonuclease activity, like for instance I-Tev-I, Col E7, NucA and Fok-I. In a more preferred embodiment, said rare-cutting endonuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-Tevl described in WO2012138927.

The invention encompasses both wild-type and variant rare-cutting endonucleases. It is understood that, rare-cutting endonuclease according to the present invention can also comprise single or plural additional amino acid substitutions or amino acid insertion or amino acid deletion introduced by mutagenesis process well known in the art. In the frame of the present invention, such variant endonucleases remain functional, i.e. they retain the capacity of recognizing and specifically cleaving a target sequence.

Are also encompassed in the scope of the present invention rare-cutting endonuclease variants which present a sequence with high percentage of identity or high percentage of homology with sequences of rare-cutting endonuclease described in the present application, at nucleotidic or polypeptidic levels. By high percentage of identity or high percentage of homology it is intended 70%, more preferably 75%, more preferably 80%, more preferably 85%, more preferably 90%, more preferably 95, more preferably 97%, more preferably 99% or any integer comprised between 70% and 99%.

To efficiently modify a specific nucleic acid sequence with algal genome, said rare-cutting endonuclease is expressed in an algal cell over several generations, preferably, more than 10², more preferably more than 10⁴, even more preferably more than 10⁶ generations. In some embodiments, said vectors encoding rare-cutting endonuclease continue to be expressed during different rounds of cell division. To maintain vector expression over several generations, efficient transient gene expression can be realized using expression vectors which require for example codon optimization and recruitment of strong promoter.

In particular embodiment, said vector encoding rare-cutting endonuclease can be integrated into algae genome and express rare-cutting endonuclease over several generations. Standard molecular biology techniques of recombinant DNA and cloning known to those skilled in the art can be applied to carry out the methods unless otherwise specified.

Finally, the cell progeny of said transfected algal cells having a modified target sequence is selected. In preferred embodiment, the method according to the present invention further comprises selecting transfected algae in which said gene encoding said rare-cutting endonuclease has been integrated into the genome. Said modified target sequence or presence of integrated gene encoding rare-cutting endonuclease within genome can be for instance identified by PCR, sequencing, southern blot assays, Northern blot and Western blot. In more preferred embodiment, few days to few weeks after transfection, cells are spread and grown on solid medium then different colonies are picked and analyzed for the presence of targeted modification by PCR, sequencing, southern blot assays, Northern blot and western blot as non limiting examples. The modification events within target sequence can also be selected by the extinction of phenotypes or by the identification of new phenotypes resulting from these modifications.

In a more preferred embodiment, the method according to the present invention further comprises selecting the algal cells that display modifications in multi-copy genes or in different alleles after one run of the method according to the present invention. Multi-copy gene or multiple allele disruptions events can be identified by PCR, sequencing, southern blot, northern blot and western blot assays as non limiting examples. Multi-copy gene or multiple allele modification can also be selected by the extinction of phenotypes or by the identification of new phenotypes these multiple gene or allele modifications.

In a particular embodiment, the present invention relates to a method comprising obtaining mosaic clones comprising cells in which said target sequence has undergone different modifications. In a preferred embodiment, mosaic clones are obtained after algal cell transfection with vectors encoding rare-cutting endonuclease and spread of said transfected algal cell on solid medium. Each clone comprises different populations of cells in which said target sequence has undergone NHEJ event or homologous recombination or is unmodified. These populations result from the rare-cutting endonuclease expression during growth of the colony. Therefore, different modifications of the target sequence can be segregated from a single clone.

Transformation methods require effective selection markers to discriminate successful transformants cells. The majority of the selectable markers include genes with a resistance to antibiotics. Therefore, vectors according to the present invention can further comprise selectable markers and said transfected algal cells are selected under selective agent. Only few publications refer to selection markers usable in Diatoms. (Dunahay, Jarvis et al. 1995; Zaslayskaia, Lippmeier et al. 2001) report the use of the neomycin phosphotransferase II (nptII), which inactivates G418 by phosphorylation, in Cyclotella cryptica, Navicula saprophila and Phaeodactylum tricornutum species. Falciatore, Casotti et al. 1999 and Zaslayskaia, Lippmeier et al. 2001 report the use of the Zeocin or Phleomycin resistance gene (Sh ble), acting by stochiometric binding, in Phaeodactylum tricornutum and Cylindrotheca fusiformis species. In Zaslayskaia, Lippmeier et al. 2001, the use of N-acetyltransferase 1 gene (Nat1) conferring the resistance to Nourseothricin by enzymatic acetylation is reported in Phaeodactylum tricornutum and Thalassiosira pseudonana. It is understood that use of the previous specific selectable markers are comprised in the scope of the present invention and that use of other genes encoding other selectable markers including, for example and without limitation, genes that participate in antibiotic resistance. In a more preferred embodiment, the vector encoding for selectable marker and the vector encoding for rare-cutting endonuclease are different vectors.

In particular embodiments, the gene encoding a rare-cutting endonuclease according to the present invention is placed under the control of a promoter. Suitable promoters include tissue specific and/or inducible promoters. Tissue specific promoters control gene expression in a tissue-dependent manner and according to the developmental stage of the algae. The transgenes driven by these type of promoters will only be expressed in tissues where the transgene product is desired, leaving the rest of the tissues in the algae unmodified by transgene expression. Tissue-specific promoters may be induced by endogenous or exogenous factors, so they can be classified as inducible promoters as well. An inducible promoter is a promoter which initiates transcription only when it is exposed to some particular (typically external) stimulus. Particularly preferred for the present invention are: a light-regulated promoter, nitrate reductase promoter, eukaryotic metallothionine promoter, which is induced by increased levels of heavy metals, prokaryotic lacZ promoter which is induced in response to isopropyl-β-D-thiogalacto-pyranoside (IPTG), steroid-responsive promoter, tetracycline-dependent promoter and eukaryotic heat shock promoter which is induced by increased temperature.

A variety of different methods are known for transfecting vectors into algal cells nuclei or chloroplasts. In various embodiments, vectors can be introduced into algae nuclei by, for example without limitation, electroporation, magnetophoresis. The latter is a nucleic acid introduction technology using the processes of magnetophoresis and nanotechnology fabrication of micro-sized linear magnets (Kuehnle et al., U.S. Pat. No. 6,706,394; 2004; Kuehnle et al., U.S. Pat. No. 5,516,670; 1996) that proved amenable to effective chloroplast engineering in freshwater Chlamydomonas, improving plastid transformation efficiency by two orders of magnitude over the state-of the-art of biolistics (Champagne et al., Magnetophoresis for pathway engineering in green cells. Metabolic engineering V: Genome to Product, Engineering Conferences International Lake Tahoe CA, Abstracts pp 76; 2004). Polyethylene glycol treatment of protoplasts is another technique that can be used to transform cells (Maliga 2004). In various embodiments, the transformation methods can be coupled with one or more methods for visualization or quantification of nucleic acid introduction to one or more algae. Direct microinjection of purified endonucleases of the present invention in algae can be considered. Also appropriate mixtures commercially available for protein transfection can be used to introduce endonucleases in algae according to the present invention. More broadly, any means known in the art to allow delivery inside cells or subcellular compartments of agents/chemicals and molecules (proteins) can be used to introduce endonucleases in algae according to the present invention including liposomal delivery means, polymeric carriers, chemical carriers, lipoplexes, polyplexes, dendrimers, nanoparticles, emulsion, natural endocytosis or phagocytose pathway as non-limiting examples. In a more preferred embodiment, said transformation construct is introduced into host cell by particle inflow gun bombardment or electroporation.

Endonucleolytic breaks are known to stimulate homologous recombination. Therefore, in particular embodiments, the present invention relates to a method to target sequence insertion (knock-in) into chosen loci of the genome.

In particular embodiments, the knock-in algae is made by transfecting said algal cell with a rare-cutting endonuclease as described above, to induce a cleavage within or adjacent to a nucleic acid target sequence, and with a donor matrix containing a transgene to introduce said transgene by a knock-in event. Said donor matrix comprises a sequence homologous to at least a portion of the target nucleic acid sequence, such that homologous recombination occurs between the target DNA sequence and the donor matrix. In particular embodiments, said donor matrix comprises first and second portions which are homologous to region 5′ and 3′ of the target nucleic acid, respectively. Said donor matrix in these embodiments also comprises a third portion positioned between the first and the second portion which comprises no homology with the regions 5′ and 3′ of the target nucleic acid sequence. Following cleavage of the target nucleic acid sequence, a homologous recombination event is stimulated between the genome containing the target nucleic acid sequence and the donor matrix. Preferably, homologous sequences of at least 50 bp, preferably more than 100 bp and more preferably more than 200 bp are used within said donor matrix. Therefore, the donor matrix is preferably from 200 bp to 6000 bp, more preferably from 1000 bp to 2000 bp. Indeed, shared DNA homologies are located in regions flanking upstream and downstream the site of the break and the DNA sequence to be introduced should be located between the two arms.

In particular embodiments, said donor matrix can comprise a positive selection marker between the two homology arms and eventually a negative selection marker upstream of the first homology arm or downstream of the second homology arm. The marker(s) allow(s) the selection of algae having inserted the sequence of interest by homologous recombination at the target site. Depending on the location of the targeted genome sequence wherein DSB event has occurred, such template can be used to knock-out a gene, e.g. when the template is located within the open reading frame of said gene, or to introduce new sequences or genes of interest. This technology further increases the exploitation potential of algae by conferring them commercially desirable traits for various biotechnological applications. Sequence insertions by using such templates can be used to modify a targeted existing gene, by correction or replacement of said gene (allele swap as a non-limiting example), or to up- or down-regulate the expression of the targeted gene (promoter swap as non-limiting example), said targeted gene correction or replacement conferring one or several commercially desirable traits.

According to a particularly advantageous embodiment, the donor matrix comprising sequences sharing homologies with the regions surrounding the targeted genomic nucleic acid cleavage site in algae as defined above is included in the vector encoding said rare-cutting endonuclease. Preferably, homologous sequences of at least 50 bp, preferably more than 100 bp and more preferably more than 200 bp are used within said donor matrix. Therefore, the donor matrix is preferably from 200 bp to 6000 bp, more preferably from 1000 bp to 2000 bp. Alternatively, the vector encoding for a rare-cutting endonuclease and the vector comprising the donor matrix are different vectors.

In a particular embodiment of the methods envisaged herein the mutagenesis is increased by transfecting the cell with a further transgene coding for a catalytic domain. In a particular embodiment, the present invention provides improved methods for ensuring targeted modification in the genetic modification of an algal cell and provides a method for increasing mutagenesis at the target nucleic acid sequence to generate at least one DNA cleavage and a loss of genetic information around said target nucleic acid sequence thus preventing any scarless re-ligation by NHEJ. In a more preferred embodiment, said catalytic domain is a DNA end-processing enzyme. Non limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise a protein domain or catalytically active derivate of the protein domain selected from the group consisting of hExol (EXO1_HUMAN), Yeast Exol (EXO1_YEAST), E. coli Exol, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In a more preferred embodiment, said catalytic domain has an exonuclease activity, in particular a 3′-5′ exonuclease activity. In a more preferred embodiment, said catalytic domain has TREX exonuclease activity, more preferably TREX2 activity. In another preferred embodiment, said catalytic domain is encoded by a single chain TREX polypeptide. In a particular embodiment, said catalytic domain is fused to the N-terminus or C-terminus of said rare-cutting endonuclease. In a more preferred embodiment, said catalytic domain is fused to said rare-cutting endonuclease by a peptide linker. Said peptide linker is a peptide sequence which allows the connection of different monomers in a fusion protein and the adoption of the correct conformation for said fusion protein activity and which does not alter the specificity of either of the monomers for their targets. Peptide linkers can be of various sizes, from 3 amino acids to 50 amino acids as a non limiting indicative range. Peptide linkers can also be structured or unstructured. It has been found that the coupling of the enzyme SCTREX2 with an endonuclease such as a meganuclease ensures high frequency of targeted mutagenesis in algal cells, such as diatoms.

In another embodiment, the present invention relates to a method for modifying target nucleic acid sequence in the plastid genome of an algal cell, comprising expressing in said algal cell, a gene encoding a rare-cutting endonuclease fused to a plastid targeting sequence required for targeting the gene product into plastid compartment. Plastid targeting sequences correspond to presequences consisting of a signal peptide followed by a transit peptide-like domain as described in Gruber, Vugrinec et al. 2007. In a more preferred embodiment, said plastid targeting sequences comprise a conserved motif namely ASAF or AFAP (Kilian and Kroth 2005). As non limiting examples, said plastid targeting sequences are selected from the group consisting of SEQ ID NO: 60 to SEQ ID NO: 140.

The present invention also encompasses a method to generate a safe algal cell that no longer carries rare-cutting endonuclease transgene in its genome after gene targeting. More particularly, in certain embodiments, the method according to the present invention comprises a further step of inactivating the gene encoding the rare-cutting endonuclease present in the genome of the modified progeny cells, in particular by cultivation of the cells without selection pressure. This loss of gene function can be correlated to loss, rearrangement, or modification of the foreign DNA sequences in the genome.

In the frame of the present invention, “algae” or “algae cells” refer to different species of algae that can be used as host for genomic modification using the rare-cutting endonuclease of the present invention. Algae are mainly photoautotrophs unified primarily by their lack of roots, leaves and other organs that characterize higher plants. Term “algae” groups, without limitation, several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochtysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochtysis, Porhyra, Pseudoanabaena, Pyramimonas, Stichococcus, Synechococcus, Synechocystis, Tetraselmis, Thalassiosira, and Trichodesmium.

In a more preferred embodiment, algae are diatoms. Diatoms are unicellular phototrophs identified by their species-specific morphology of their amorphous silica cell wall, which vary from each other at the nanometer scale. Diatoms includes as non limiting examples: Phaeodactylum, Fragilariopsis, Thalassiosira, Coscinodiscus, Arachnoidiscusm, Aster omphalus, Navicula, Chaetoceros, Chorethron, Cylindrotheca fusiformis, Cyclotella, Lampriscus, Gyrosigma, Achnanthes, Cocconeis, Nitzschia, Amphora, and Odontella.

In another aspect, also encompassed in the scope of the present invention, a genetically modified algal cell is provided obtained or obtainable by the methods described above. In particular embodiments, such genetically modified algal cells are characterized by the presence of a sequence encoding a rare-cutting endonuclease transgene and a modification in a targeted gene.

Particularly, is comprised in the scope of the invention, a genetically modified algal cell characterized in that its genome comprise a targeted modification in more than one allele and/or in multiple copy or homologous genes. More particularly, is comprised in the scope of the present invention, a genetically modified algal cell characterized in that its genome comprise transgenes encoding a TALE-Nuclease, a TALE-Nuclease and a TREX exonuclease or a meganuclease and a TREX exonuclease. The present invention also relates a genetically modified algal cell characterized in that its genome comprises a TALE-Nuclease-induced targeted modification. In a particular embodiment, genetically modified algal cells are provided of which the genome includes a gene encoding a rare-cutting endonuclease which expression is under control of inducible promoter.

Using the method described above, the inventor succeeded to generate diatoms in which endogenous genes were inactivated using TALE-nuclease. By inactivated, it is meant, that the gene encodes a non-functional protein or does not express the protein. Inactivating a gene can be the consequence of a mutation in the gene, for instance a deletion, a substitution, or an addition of at least one nucleotide. The gene can also be inactivated by the insertion of a transgene in the gene of interest, particularly, by homologous recombination. The transgene can encode for a non functional form of the protein.

Two genes involved in lipid metabolism: UDP-glucose pyrophosphorylase (UGPase) and putative elongase gene were inactivated in diatom strains using specific TALE-nuclease to increase lipid content. The UDP-glucose pyrophosphorylase gene encodes for an enzyme involved in lipid metabolism, particularly in the metabolic pathway leading to the accumulation of energy-rich storage compounds, such as chrysolaminarin (μ-1, 3-glucan). The putative elongase gene is an enzyme involved in the carbon length of the fatty acids.

Thus, the present invention relates to a genetically modified algal cell in which UDP-glucose pyrophosphorylase (UGPase) gene is inactivated, particularly the UDP-glucose pyrophosphorylase gene has at least 70%, preferably 75%, 80%, 85%, 90%, 95% identity with the sequence SEQ ID NO: 41. In a more particular embodiment, the genetically modified algal cell in which UGPase is inactivated has been obtained using TALE-nuclease, preferably TALE-nuclease which targets a sequence within the UGPase gene, more particularly a target sequence SEQ ID NO: 44.

In another aspect, the present invention relates to a genetically modified algal cell in which putative elongase gene is inactivated, particularly the putative elongase gene has at least 70%, preferably 75%, 80%, 85%, 90%, 95% identity with the sequence SEQ ID NO: 52. In a more particular embodiment, the genetically modified algal cell in which putative elongase is inactivated has been obtained using TALE-nuclease, preferably TALE-nuclease which targets a sequence within the putative elongase gene, more particularly a target sequence SEQ ID NO: 55.

In particular embodiment, said genetically modified algal cell is a diatom, more preferably a Phaeodactylum tricornutum or a Thalassiosira pseudonana. In a particular embodiment, said genetically modified diatoms are Phaeodactylum tricornutum strains deposited within the Culture Collection of Algae and Protozoa (CCAP, Scottish Marine Institute, Oban, Argyll PA34 1QA, Scotland), on May 29^{th}, 2013, under depositor's strain numbers pt-37-7A1 and pt-42-11B5. These strains have received acceptance numbers CCAP 1055/12 with respect to pt-37-7A1 and CCAP 1055/13 with respect to pt-42-11B5.

### DEFINITIONS

By “gene” it is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns and a 3′ untranslated region. The gene may further be comprised of terminators, enhancers and/or silencers.

By “genome” it is meant the entire genetic material contained in a cell such as nuclear genome, chloroplastic genome, mitochondrial genome.

As used herein, the term “locus” is the specific physical location of a DNA sequence (e.g. of a gene) on a nuclear, mitochondria or choloroplast genome. As used in this specification, the term “locus” usually refers to the specific physical location of an endonuclease's target sequence. Such a locus, which comprises a target sequence that is recognized and cleaved by an endonuclease according to the invention, is referred to as “locus according to the invention”.

By “target sequence” is intended a polynucleotide sequence that can be processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria or chloroplasts as non-limiting examples. The nucleic acid target sequence is defined by the 5′ to 3′ sequence of one strand of said target.

As used herein, the term “transgene” refers to a sequence inserted at in an algal genome. Preferably, it refers to a sequence encoding a polypeptide. Preferably, the polypeptide encoded by the transgene is either not expressed, or expressed but not biologically active, in the algae or algal cells in which the transgene is inserted. Most preferably, the transgene encodes a polypeptide useful for increasing the usability and the commercial value of algae. Also, the transgene can be a sequence inserted in an algae genome for producing an interfering RNA.

By “homologous” it is meant a sequence with enough identity to another one to lead to homologous recombination between sequences, more particularly having at least 95% identity, preferably 97% identity and more preferably 99%.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

By “phenotype” it is meant an algae's or a algae cell's observable traits. The phenotype includes viability, growth, resistance or sensitivity to various marker genes, environmental and chemical signals, etc. . . . .

By “vector” is intended to mean a nucleic acid molecule capable of transporting another nucleic acid to which it has been linked. A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available. Some useful vectors include, for example without limitation, pGEM13z. pGEMT and pGEMTEasy {Promega, Madison, Wis.); pSTBluel (EMD Chemicals Inc. San Diego, Calif.); and pcDNA3.1, pCR4-TOPO, pCR-TOPO-II, pCRBlunt-II-TOPO (Invitrogen, Carlsbad, Calif.). Preferably said vectors are expression vectors, wherein the sequence(s) encoding the rare-cutting endonuclease of the invention is placed under control of appropriate transcriptional and translational control elements to permit production or synthesis of said rare-cutting endonuclease. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said rare-cutting endonuclease is a heterodimer, the two polynucleotides encoding each of the monomers are included in two vectors to avoid intraplasmidic recombination events. In another embodiment the two polynucleotides encoding each of the monomers are included in one vector which is able to drive the expression of both polynucleotides, simultaneously. In some embodiments, the vector for the expression of the rare-cutting endonucleases according to the invention can be operably linked to an algal-specific promoter. In some embodiments, the algal-specific promoter is an inducible promoter. In some embodiments, the algal-specific promoter is a constitutive promoter. Promoters that can be used include, for example without limitation, a Pptca1 promoter (the CO2 responsive promoter of the chloroplastic carbonic anyhydrase gene, ptca1, from P. tricornutum), a NITI promoter, an AMTI promoter, an AMT2 promoter, an AMT4 promoter, a RHI promoter, a cauliflower mosaic virus 35S promoter, a tobacco mosaic virus promoter, a simian virus 40 promoter, a ubiquitin promoter, a PBCV-I VP54 promoter, or functional fragments thereof, or any other suitable promoter sequence known to those skilled in the art. In another more preferred embodiment according to the present invention the vector is a shuttle vector, which can both propagate in E. coli (the construct containing an appropriate selectable marker and origin of replication) and be compatible for propagation or integration in the genome of the selected algae.

The term “promoter” as used herein refers to a minimal nucleic acid sequence sufficient to direct transcription of a nucleic acid sequence to which it is operably linked. The term “promoter” is also meant to encompass those promoter elements sufficient for promoter-dependent gene expression controllable for cell-type specific expression, tissue specific expression, or inducible by external signals or agents; such elements may be located in the 5′ or 3′ regions of the naturally-occurring gene.

By “inducible promoter” it is mean a promoter that is transcriptionally active when bound to a transcriptional activator, which in turn is activated under a specific condition(s), e.g., in the presence of a particular chemical signal or combination of chemical signals that affect binding of the transcriptional activator, e.g., CO₂ or NO₂, to the inducible promoter and/or affect function of the transcriptional activator itself.

The term “transfection” or “transformation” as used herein refer to a permanent or transient genetic change, preferably a permanent genetic change, induced in a cell following incorporation of non-host nucleic acid sequences.

The term “host cell” refers to a cell that is transformed using the methods of the invention. In general, host cell as used herein means an algal cell into which a nucleic acid target sequence has been modified.

By “catalytic domain” is intended the protein domain or module of an enzyme containing the active site of said enzyme; by active site is intended the part of said enzyme at which catalysis of the substrate occurs. Enzymes, but also their catalytic domains, are classified and named according to the reaction they catalyze. The Enzyme Commission number (EC number) is a numerical classification scheme for enzymes, based on the chemical reactions they catalyze (http://www.chem.qmul.ac.uk/iubmb/enzyme/).

By “mutagenesis” is understood the elimination or addition of at least one given DNA fragment (at least one nucleotide) or sequence, bordering the recognition sites of rare-cutting endonuclease.

By “NHEJ” (non-homologous end joining) is intended a pathway that repairs double-strand breaks in DNA in which the break ends are ligated directly without the need for a homologous template. NHEJ comprises at least two different processes. Mechanisms involve rejoining of what remains of the two DNA ends through direct re-ligation (Critchlow and Jackson 1998) or via the so-called microhomology-mediated end joining (Ma, Kim et al. 2003) that results in small insertions or deletions and can be used for the creation of specific gene knockouts.

The term “Homologous recombination” refers to the conserved DNA maintenance pathway involved in the repair of DSBs and other DNA lesions. In gene targeting experiments, the exchange of genetic information is promoted between an endogenous chromosomal sequence and an exogenous DNA construct. Depending of the design of the targeted construct, genes could be knocked out, knocked in, replaced, corrected or mutated, in a rational, precise and efficient manner. The process requires homology between the targeting construct and the targeted locus. Preferably, homologous recombination is performed using two flanking sequences having identity with the endogenous sequence in order to make more precise integration as described in WO9011354.

By “Mosaic clone” is intended clone that comprises cells in which said target sequence has undergone different modifications. Each clone comprises different populations of cells in which said target sequence has undergone NHEJ event or homologous recombination or is unmodified. These populations result from the rare-cutting endonuclease expression during growth of the colony. Therefore, different modifications of the target sequence can be segregated from a single clone.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of”, “chosen from” and the like include mixtures of the specified materials.

Where a numerical limit or range is stated herein, the endpoints are included. Also, all values and sub-ranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Increase of Targeted Mutagenesis Frequency at Endogenous Locus Using the PTRI20 Meganuclease

To investigate the ability of one meganuclease to increase the targeted mutagenesis frequency at diatom endogenous locus, one engineered meganuclease, called PTRI20 encoded by the pCLS17038 plasmid (SEQ ID NO: 1) designed to cleave the DNA sequence 5′-GTTTTACGTTGTACGACGTCTAGC-3′ (SEQ ID NO: 2) was created. The meganuclease encoding plasmid was co-transformed with plasmid encoding selection gene (Nat1) (SEQ ID NO: 3) into diatoms. The mutagenesis rate was measured by deep sequencing on individual clones resulting from transformations.

### Materials and Methods

### Culture Conditions

Phaeodactylum tricornutum Bohlin clone CCMP2561 was grown in filtered Guillard's f/2 medium without silica (40°/° ° w/v Sigma Sea Salts S9883), supplemented with 1× Guillard's f/2 marine water enrichment solution (Sigma G0154) in a Sanyo incubator (model MLR-351) at a constant temperature (20+/−0.5° C.). The incubator is equipped with white cold neon light tubes that produce an illumination of about 120 μmol photons m⁻² s⁻¹ and a photoperiod of 12 h light: 12 h darkness (illumination period from 9 AM to 9 PM). Liquid cultures were made in ventilated cap flasks put on an orbital shaker (Polymax 1040) at a frequency of 30 revolutions min⁻¹ and an angle of 5°.

### Genetic Transformation

5.10⁷ cells were collected from exponentially growing liquid cultures (concentration about 10⁶ cells/ml) by centrifugation (3000 rpm for 10 minutes at 20° C.). The supernatant was discarded and the cell pellet resuspended in 500 μl of fresh f/2 medium. The cell suspension was then spread on the center one-third of a 10 cm 1% agar plate containing 20°/° ° sea salts supplemented with f/2 solution without silica. Two hours later, transformation was carried out using the biolistic technology (Biolistic PDS-1000/He Particle Delivery System (BioRad)). The protocol is adapted from Apt, Kroth-Pancic et al. 1996 and Falciatore, Casotti et al. 1999 with minor modifications. Briefly, M17 tungstene particles (1.1 μm diameter, BioRad) were coated with 9 μg of total amount of DNA containing 3 μg of meganuclease encoding plasmid (pCLS17038), 3 μg nat1 selection plasmid (pCLS16604) (SEQ ID NO: 3) and 3 μg of empty vector (pCLS0003) (SEQ ID NO: 4) using 1.25M CaCl2 and 20 mM spermidine according to the manufacturer's instructions. As negative control, beads were coated with a DNA mixture containing 3 μg Nat1 selection plasmid (pCLS16604) and 6 μg empty vector (pCLS0003). Agar plates with the diatoms to be transformed were positioned at 7.5 cm from the stopping screen within the bombardment chamber (target shelf on position two). A burst pressure of 1550 psi and a vacuum of 25 Hg/in were used. After bombardment, plates were incubated for 48 hours with a 12 h light: 12 h dark photoperiod.

### Selection

Two days post transformation, bombarded cells were gently scrapped with 700 μl of f/2 medium without silica and spread on two 10 cm 1% agar plates (20°/° ° sea salts supplemented with f/2 medium without silica) containing 300 μg ml⁻¹ nourseothricin (Werner Bioagents). Plates were then placed in the incubator under a 12 h light: 12 h darkness cycle for at least three weeks. 3 to 4 weeks later, on average, emerging clones resulting from the stable transformation were re-streaked on fresh 10 cm 1% agar plates containing 300 μg ml⁻¹ nourseothricin.

### Characterization

### Measure of the Mutagenesis Frequency by Deep Sequencing

Resistant colonies were picked and dissociated in 20 μl of lysis buffer (1% TritonX-100, 20 mM Tris-HCl pH8, 2 mM EDTA) in an eppendorf tube. Tubes were vortexed for at least 30 sec and then kept on ice for 15 min. After heating for 10 min at 85° C., tubes were cooled down at RT and briefly centrifuged to pellet cells debris. Supernatants were used immediately or stocked at 4° C. 5 μl of a 1:5 dilution in milliQ H2O of the supernatant, were used for PCR reactions. The PTRI20 target was amplified using specific primers flanked by adaptators needed for HTS sequencing on the 454 sequencing system (454 Life Sciences) using the primer

PTRI20_For1
(SEQ ID NO: 5)
5′- CCATCTCATCCCTGCGTGTCTCCGACTCAG-TAG-
CGGTTGTCATGGATAGCGGAGC -3′
and
PTRI20_Rev1
(SEQ ID NO: 6)
5′- CCTATCCCCTGTGTGCCTTGGCAGTCTCAG-
CCCCAGACGATTCGAAGTCGTCC -3′.

The PCR products were purified on magnetic beads (Agencourt AMPure XP, Beckman Coulter). 5000 to 10 000 sequences per sample were analyzed.

### Results

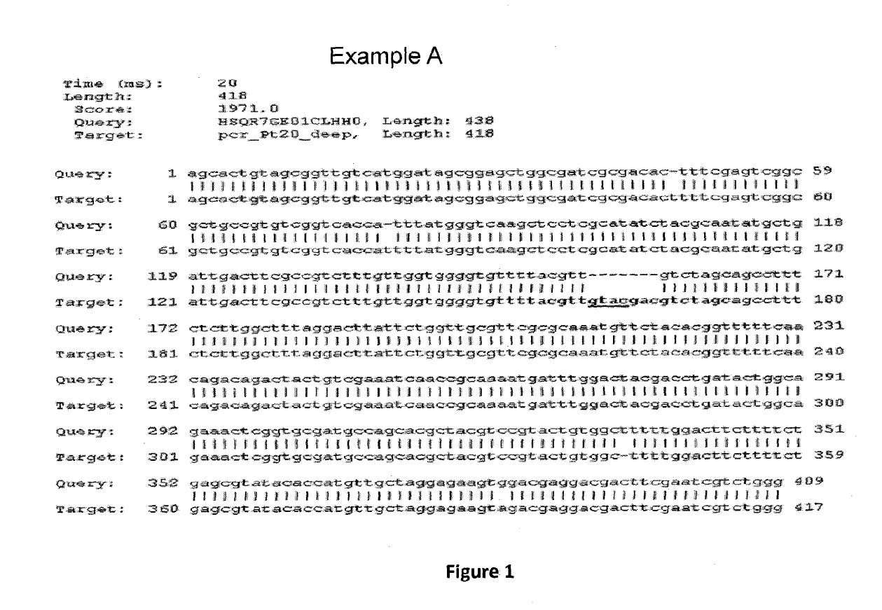

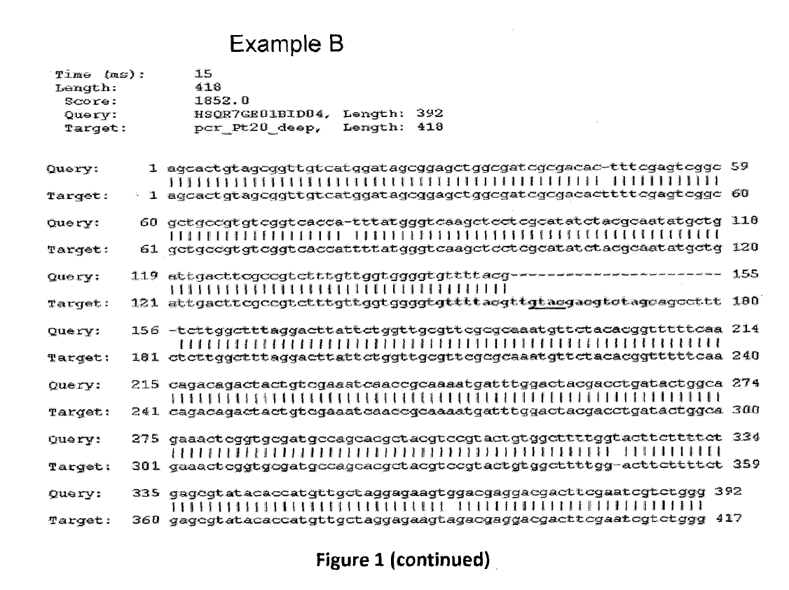

Several weeks after the transformation of diatoms with meganuclease PTRI20 (condition 1), few clones are obtained. One clone was selected to measure the mutagenesis frequency induced by the PTRI20 meganuclease, a lysis of this clone was done and the mutagenesis frequency was determined by deep sequencing (Tablet). In parallel, we analyzed 2 clones resulting from the transformation with the empty vector (condition 2). Whereas, we observed 0.032% (3/9446) of PCR fragments carrying a mutation in the sample corresponding to the clone transformed with PTRI20, we did not detected any mutagenic event when the diatoms were transformed with the empty vector. Examples of mutagenic events found in the sample corresponding to PTRI20 conditions are presented in FIG. 1.

Thus, the PTRI20 meganuclease was able to induce targeted mutagenesis events at the endogenous locus in diatoms.

TABLE 1
Mutagenesis-induced by PTRI20 meganuclease.

| | | % Targeted Mutagenesis |
|---|---|---|
| | Clone | (Nb mutated sequences/ |
| Diatoms Transformed with | Number | Nb Total sequences) |
| PTRI20 (Condition 1) | 1 | 0.032% (3/9446) |
| Empty vector (Condition 2) | 1 | 0 |
| | 2 | 0 |

A lysis of the clones resulting from the transformation with the meganuclease (condition 1) or from transformation with the empty vector (condition 2) was done. A PCR surrounding the PTRI20 target was performed and the percentage of the mutagenesis frequency induced by the PTRI20 meganuclease was determined by deep sequencing analysis of amplicons surrounding the specific target.

### Example 2

### High Targeted Mutagenesis Frequency at Endogenous Locus of Diatoms Using the Combination of SCTREX2 and PTRI20 Meganuclease

To investigate the ability of the DNA processing enzyme single chain TREX2 (SCTREX2) to increase the targeted mutagenesis frequency induced by a meganuclease, one engineered meganuclease, called PTRI20 encoded by the pCLS17038 plasmid (SEQ ID NO: 1) designed to cleave the DNA 5′-GTTTTACGTTGTACGACGTCTAGC-3′ (SEQ ID NO: 2) was used. This meganuclease was co-transformed with a plasmid encoding selection gene (Nat1) (NAT) (SEQ ID NO: 3) and with a plasmid encoding a DNA processing enzyme, called SCTREX2 encoded by the pCLS18296 (SEQ ID NO: 7). The mutagenesis rate was visualized by T7 assay and measured by Deep sequencing on individual clones resulting from transformation.

### Materials and Methods

Phaeodactylum tricornutum Bohlin clone CCMP2561 was grown and transformed according to the method described in example 1 with M17 tungstene particles (1.1 μm diameter, BioRad) coated with 9 μg of total amount of DNA containing 3 μg of meganuclease encoding plasmid (pCLS17038), 3 μg SCTREX2 (pCLS18296) and 3 μg Nat1 selection plasmid (pCLS16604) (SEQ ID NO: 3) (Condition 1) using 1.25M CaCl2 and 20 mM spermidine according to the manufacturer's instructions. As negative controls, beads were coated with a DNA mixture containing 3 μg of meganuclease encoding plasmid pCLS17038, 3 μg Nat1 selection plasmid (pCLS16604) and 3 μg empty vector (pCLS0003) (Condition 2) or 3 μg Nat1 selection plasmid (pCLS16604) and 6 μg empty vector (pCLS0003) (SEQ ID NO: 4) (Condition 3).

### Characterization

### A-Colony Screening

After selection, resistant colonies were picked and dissociated according to the method described in example 1. Supernatants were used for each PCR reaction. Specific primers for meganuclease screen: meganuclease_For1 5′-TTAACAATTGAATCTCGCCTATTCATGGTG-3′ (SEQ ID NO: 8) and meganuclease_Rev1 5′-TAGCGCTCGAGTTACTAAGGAGAGGACTTTTTCTT-3′ (SEQ ID NO: 9), for SCTREX2 screen SCTREX2_For1 5′-AATCTCGCCTATTCATGGTG-3′ (SEQ ID NO: 10) and SCTREX2_Rev1 5′-CCAGACCGGTCTGTGGAGGAG-3′ (SEQ ID NO: 11).

### B-Measure of the Mutagenesis Frequency by T7 Endonuclease Assay

PCR amplification of the PTRI20 locus was obtained with Deep sequencing primers (see list of forward and reverse primer sequences below) and genomic DNA from the colony extracts. PCR amplicons were centered on the nuclease targets and 400-500 bp long, on average.

The PCR products were purified on magnetic beads (Agencourt AMPure XP, Beckman Coulter) and quantified with a NanoDrop 1000 spectrophotometer (Thermo Scientific). 50 ng of the amplicons were denatured and then annealed in 10 μl of annealing buffer (10 mM Tris-HCl pH8, 100 mM NaCl, 1 mM EDTA) using an Eppendorf MasterCycle gradient PCR machine. The annealing program is as follows: 95° C. for 10 min; fast cooling to 85° C. at 3° C./sec; and slow cooling to 25° C. at 0.3° C./sec. The totality of the annealed DNA was digested for 15 min at 37° C. with 0.5 μl of the T7 Endonuclease I (10 U/μl) (M0302 Biolabs) in a final volume of 20 μl (1×NEB buffer 2, Biolabs). 10 μl of the digestion were then loaded on a 10% polyacrylamide MiniProtean TBE precast gel (BioRad). After migration the gel was stained with SYBRgreen and scanned on a Gel Doc XR+ apparatus (BioRad).

### C-Measure of the Mutagenesis Frequency by Deep Sequencing

The PTRI20 target was amplified with specific primers flanked by adaptator needed for HIS sequencing on the 454 sequencing system (454 Life Sciences) using the primer

PTRI20_For1
(SEQ ID NO: 5)
5′- CCATCTCATCCCTGCGTGTCTCCGACTCAG-TAG-
CGGTTGTCATGGATAGCGGAGC -3′
and
PTRI20_Rev1
(SEQ ID NO: 6)
5′- CCTATCCCCTGTGTGCCTTGGCAGTCTCAG-
CCCCAGACGATTCGAAGTCGTCC -3′.

5000 to 10 000 sequences per sample were analyzed.

### Results

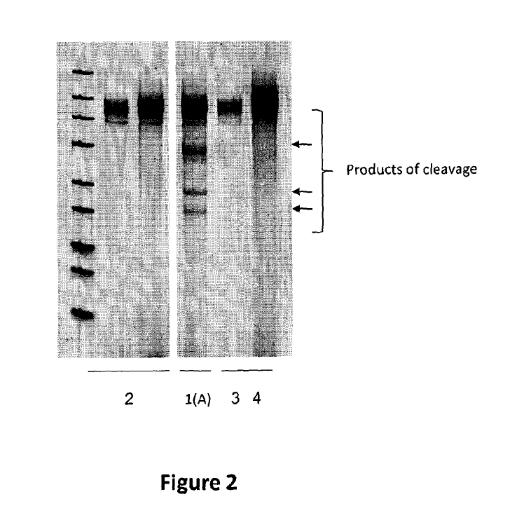

Few weeks after the transformation of diatoms with the PTRI20 meganuclease and the SCTREX2 DNA processing enzyme, 9 clones were obtained (Condition 1). Among them, 2 were positive for the presence of the meganuclease DNA sequence, 3 for the presence of the SCTREX2 only and one (called A) was positive for both transgenes which represent a rate of co-transformation around 11%. In the same time, 14 clones resulting from the transformation with the PTRI20 meganuclease alone were obtained (Condition 2). Among them, 11 were positive for the presence of meganuclease DNA sequence. Finally, 7 clones resulting from the transformation with the empty vector were obtained (Table 1) (Condition 3). In order to measure the mutagenesis frequency induced by the PTRI20 meganuclease in presence or absence of the SCTREX2 molecule, lysis from positive clone was done and the mutagenesis was determined by T7 assay and quantified by Deep sequencing (FIG. 2).

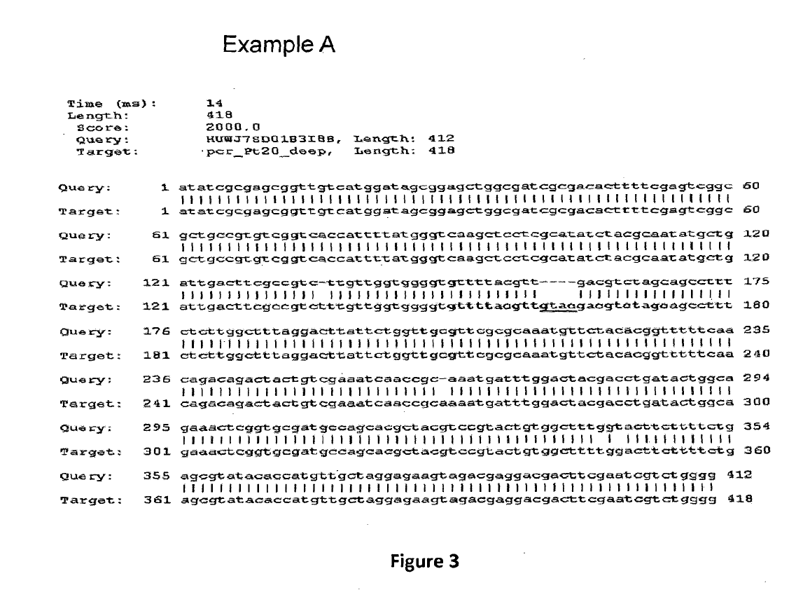

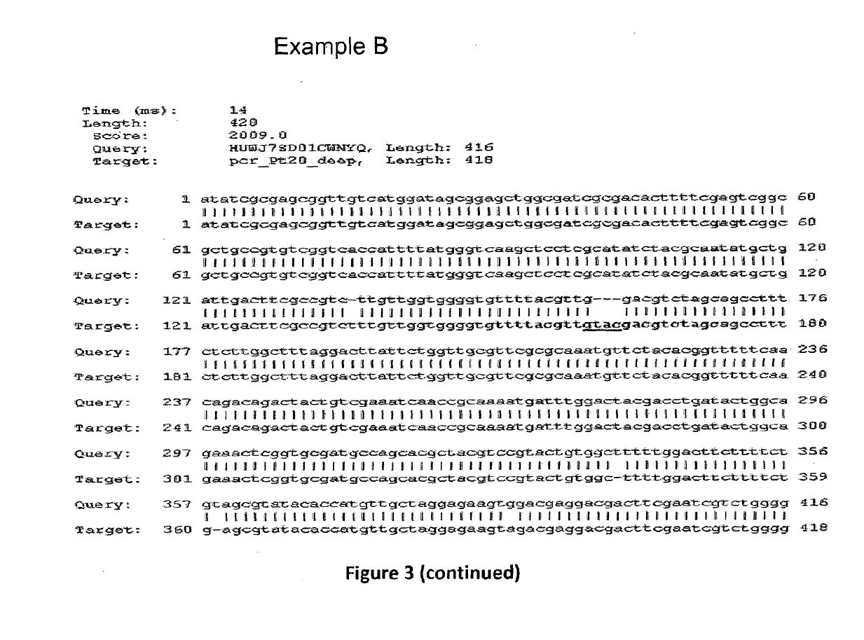

The clone (A) corresponding to the positive clone for both meganuclease and SCTREX2 DNA sequences was tested in T7 assay. In parallel, Phaeodactylum tricornutum strain as well as the unique clone resulting from the transformation with the empty vector were also tested (FIG. 2). The clone A was positive in T7 assay which reflects the presence of mutagenic events. Due to the lack of the sensitivity of the T7 assay, no signal could be detected in the 2 clones corresponding to the diatoms transformed with the PTRI20 meganuclease alone. The mutagenesis frequency in the clone (A) was quantified by Deep sequencing analysis. Whereas, in this clone 6.9% (183/2475) of PCR fragments carried a mutation, we did not detect mutagenic event in 3 samples corresponding to diatoms transformed with the empty vector. Some examples of mutagenic events are presented in FIG. 3.

Thus, the coupling of the DNA processing enzyme SCTREX2 with a meganuclease (PTRI20) is able to cleave an endogenous target (see example 1), enhances the targeted mutagenesis frequency in diatoms (up to 6.9%).

TABLE 2
Number of clones obtained after transformation, number
of clones that have integrated the PTRI020 meganuclease
and SCTREX2 DNA sequences and the number of clones
tested in the T7 assay and Deep sequencing analysis.

| | PTRI20 + | | Empty |
|---|---|---|---|
| | SCTREX2 | PTRI20 | vector |
| Transformation condition | (Condition1) | (Condition2) | (Condition3) |
| Number of clones obtained | 9 | 14 | 7 |
| Number of clones positive for | 2 | 11 | ND |
| Meganuclease DNA sequence | | | |
| Number of clones positive for | 3 | ND | ND |
| SCTREX2 sequence | | | |
| Number of clones positive for | 1 (Called A) | ND | ND |
| presence of both transgenes | | | |
| (SCTREX2 and Meganucle- | | | |
| ase) | | | |
| Number of clones analyzed in | 1 (Called A) | 2 | 1 |
| T7 assay | | | |
| Number of clones analyzed in | 1 (Called A) | ND | 3 |
| Deep sequencing | | | |

### Example 3

### High Targeted Mutagenesis Frequency at Diatom Endogenous Locus Using the Combination SCTREX2 and PTRI02 Meganuclease

To investigate the ability of the DNA processing enzyme SCTREX2 to increase the targeted mutagenesis frequency induced by a meganuclease, one engineered meganuclease, called PTRI02 encoded by the pCLS17181 plasmid (SEQ ID NO: 12) designed to cleave the DNA sequence 5′ TTTTGACGTCGTACGGTGTCTCCG-3′ (SEQ ID NO: 13) was used. This meganuclease encoding plasmid was co-transformed with plasmid encoding selection gene (Nat1) (SEQ ID NO: 3) and with a plasmid encoding the DNA processing enzyme, SCTREX2 encoded by the pCLS18296 (SEQ ID NO: 7). The mutagenesis rate was measured by Deep sequencing on individual clones resulting from transformations.

### Materials and Methods

Phaeodactylum tricornutum Bohlin clone CCMP2561 was grown and transformed according to the method described in example 1 with M17 tungstene particles (1.1 μm diameter, BioRad) coated with 9 μg of total amount of DNA containing 3 μg of meganuclease encoding plasmid (pCLS17181), 3 μg SCTREX2 (pCLS18296) and 3 μg Nat1 selection plasmid (pCLS16604) (SEQ ID NO: 3) using 1.25M CaCl2 and 20 mM spermidine according to the manufacturer's instructions. As negative control, beads were coated with a DNA mixture containing 3 μg Nat1 selection plasmid (pCLS16604) and 6 μg empty vector (pCLS0003) (SEQ ID NO: 4).

### Characterization

### A-Colony Screening

After selection, resistant colonies were picked and dissociated according to the method described in example 1. Supernatants were used for each PCR reaction. Specific primers for meganuclease screen: meganuclease_For1 5′-TTAACAATTGAATCTCGCCTATTCATGGTG-3′ (SEQ ID NO: 8) and meganuclease_Rev1 5′-TAGCGCTCGAGTTACTAAGGAGAGGACTTTTTCTT-3′ (SEQ ID NO: 9), for SCTREX2 screen SCTREX2_For1 5′-AATCTCGCCTATTCATGGTG-3′ (SEQ ID NO: 10) and SCTREX2_Rev1 5′-CCAGACCGGTCTGTGGAGGAG-3′ (SEQ ID NO: 11).

### B-Measure of the Mutagenesis Frequency by Deep Sequencing

The PTRI02 target was amplified using a 1:5 dilution of the lysis colony with specific primers flanked by specific adaptator needed for HTS sequencing on the 454 sequencing system (454 Life Sciences) using the primer

PTRI02_For1
(SEQ ID NO: 14)
5′- CCATCTCATCCCTGCGTGTCTCCGACTCAG-TAG-
TCAGCTCCATTGGAATGTTGGC -3′
and
PTRI02_Rev1
(SEQ ID NO: 15)
5′ - CCTATCCCCTGTGTGCCTTGGCAGTCTCAG-
CCCTCCGACCAGGGAACTTACTC -3′.

The PCR products were purified on magnetic beads (Agencourt AMPure XP, Beckman Coulter). 5000 to 10 000 sequences per sample were analyzed.

### Results

Few weeks after the transformation of diatoms with both the PTRI02 meganuclease and the SCTREX2 DNA processing enzyme encoding plasmids, 7 clones were obtained. Among them, 5 were positive in PCR for the presence of both transgenes which represents a rate of co-transformation around 71%. In the same time, 7 clones resulting from the transformation with the empty vector were obtained. The mutagenesis frequency induced by the PTRI02 meganuclease in the presence of the SCTREX2 molecule was measured by Deep sequencing analysis of amplicons surrounding the PTRI02 specific target.

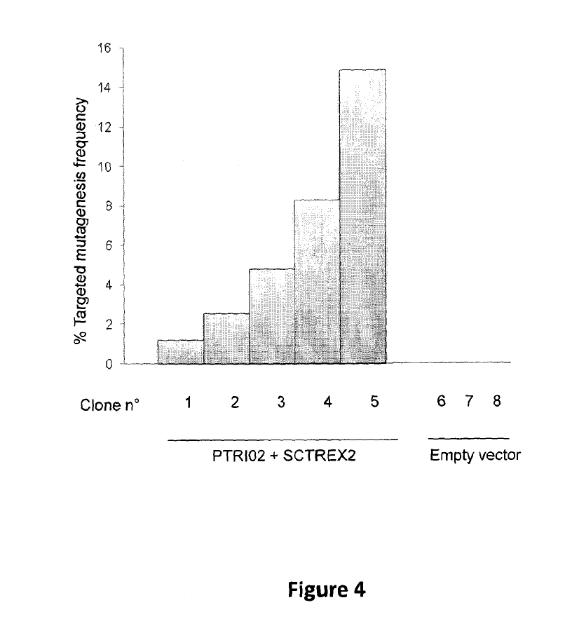

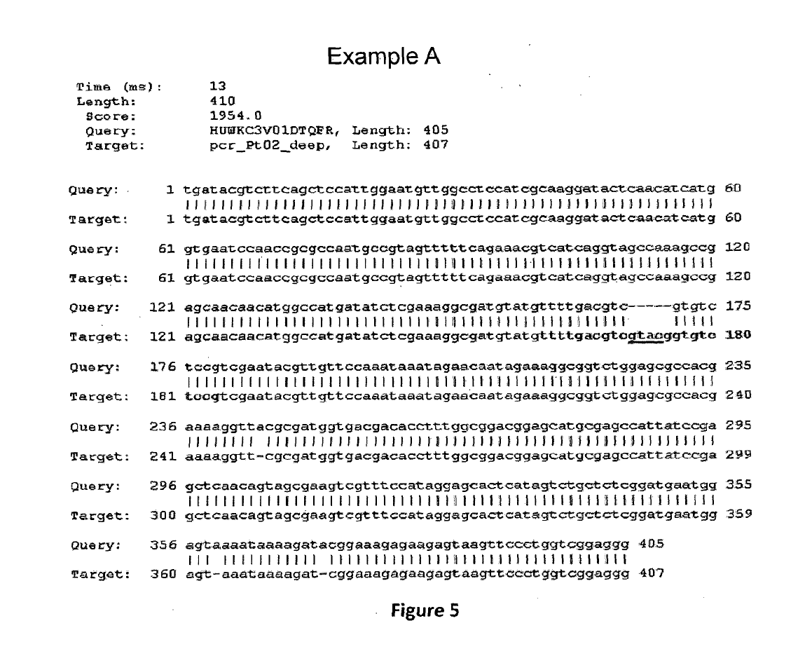

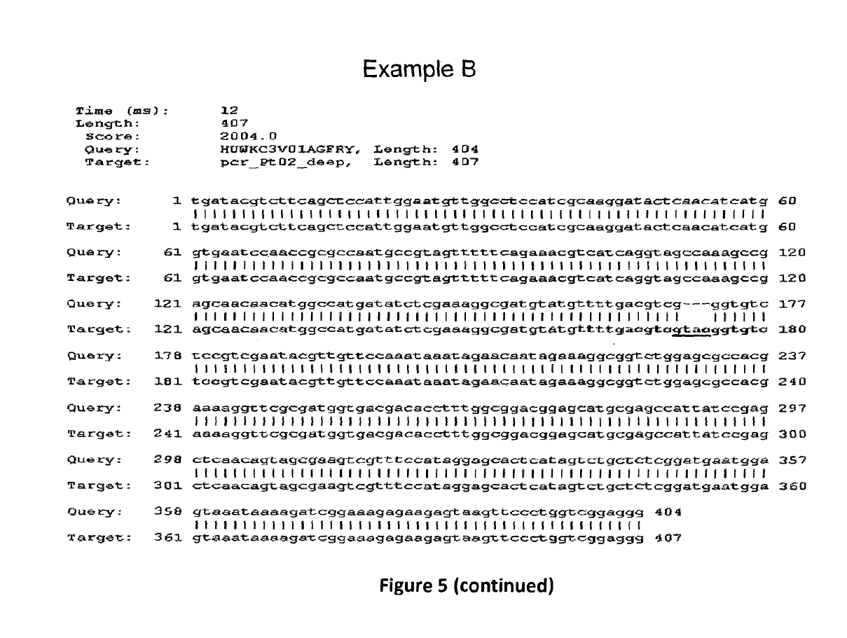

Results of the mutagenesis frequency induced by the meganuclease in presence of SCTREX2 are presented in FIG. 4. Whereas the samples corresponding to the 5 positive clones (meganuclease and SCTREX2 positive) present 1.2, 2.5, 4.8, 8.3 and 14.9% of mutated PCR fragments respectively, we did not detected any mutagenic event in the 3 samples tested corresponding to diatoms transformed with the empty vector. Thus, the 5 analyzed clones present high rates of mutagenic events. Some examples of mutagenic events are presented in FIG. 5.

To conclude, the coupling of the DNA processing enzyme SCTREX2 with one meganuclease able to cleave an endogenous target allows us to obtain high frequency of targeted mutagenesis in diatoms (up to 14%).

### Example 4

### High Targeted Mutagenesis Frequency Induced Using TALE-Nuclease Targeting Reporter Gene Stably Integrated in Diatom Genome

To investigate the ability of a TALE-Nuclease to induce targeted mutagenesis in diatoms, one engineered TALE-Nuclease, called YFP_TALE-Nuclease encoded by the pCLS17205 (SEQ ID NO: 16) and pCLS17208 (SEQ ID NO: 17) plasmids designed to cleave the DNA sequence 5′-TGAACCGCATCGAGCTGaagggcatcgacTTCAAGGAGGACGGCAA-3′ (SEQ ID NO: 18) were used. These TALE-Nuclease encoding plasmids were co-transformed with a plasmid encoding selection gene (Nat1) into a diatom strain carrying the YFP reporter gene integrated stably in multiple copies in the genome. The mutagenesis frequency induced by the designated TALE-Nuclease was measured by Deep sequencing on individual clones resulting from transformations.

### Materials and Methods

Phaeodactylum tricornutum Bohlin clone CCMP2561 was grown and transformed according to the method described in example 1 with M17 tungstene particles (1.1 μm diameter, BioRad) coated with 9 μg of total amount of DNA containing 3 μg of each monomer of TALE-Nucleases (pCLS17205 and pCLS17208) and 3 μg Nat1 (pCLS16604) (SEQ ID NO: 3) selection plasmid using 1.25M CaCl2 and 20 mM spermidine according to the manufacturer's instructions. As negative control, beads were coated with a DNA mixture containing 3 μg Nat1 selection plasmid (pCLS16604) and 6 μg empty vector (pCLS0003) (SEQ ID NO: 4).

### Characterization

### Measure of the Mutagenesis Frequency by Deep Sequencing

After selection, the genomic DNA was extracted using ZR genomic DNA (Zymo Research) Kit and the mutagenesis frequency was determined by Deep sequencing. The YFP target was amplified using a 1:7 dilution of genomic DNA, with specific primers flanked by adaptators needed for HTS sequencing on the 454 sequencing system (454 Life Sciences) using the primers

YFP_For
(SEQ ID NO: 19)
5′-CCATCTCATCCCTGCGTGTCTCCGACTCAG-Tag-
CTGCACCACCGGCAAGCTGCC-3′
and
YFP_Rev
(SEQ ID NO: 20)
5′-CCTATCCCCTGTGTGCCTTGGCAGTCTCAG-
CCTCGATGTTGTGGCGG-3′.

The PCR products were purified on magnetic beads (Agencourt AMPure XP, Beckman Coulter). 5000 to 10 000 sequences per sample were analyzed.

### Results

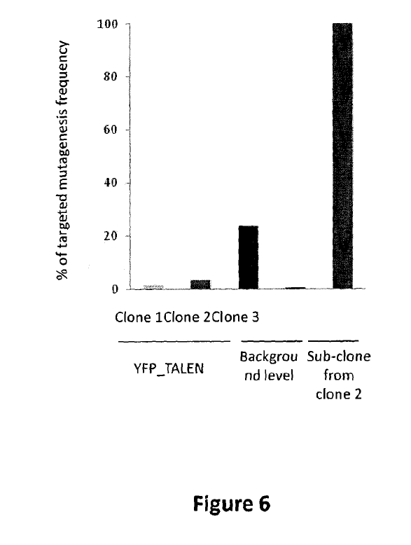

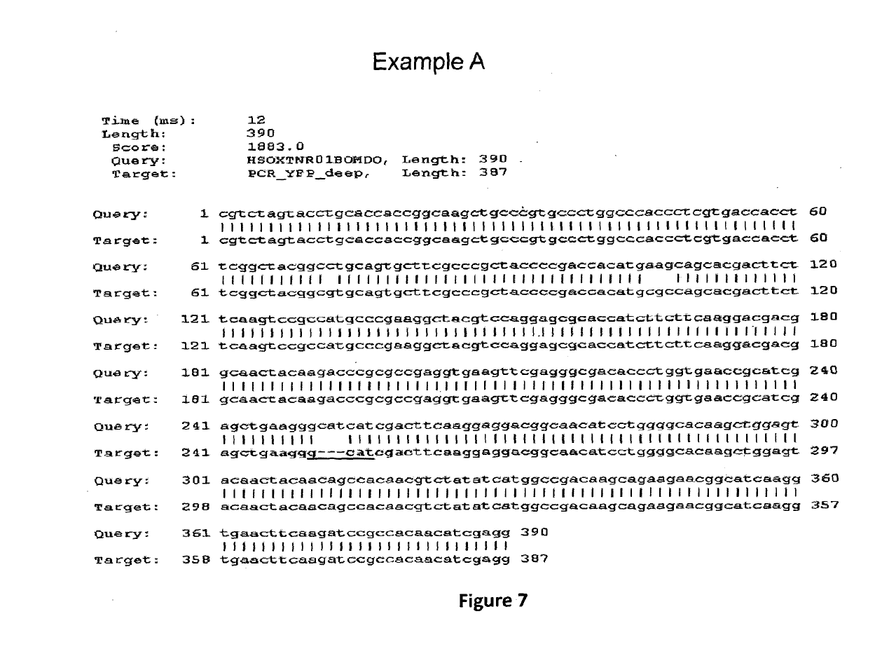

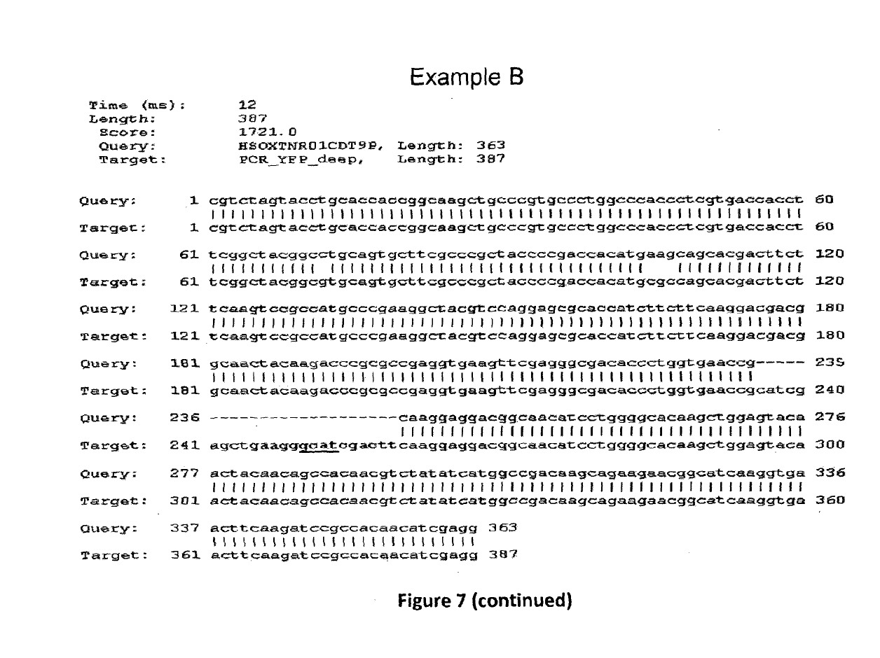

Few weeks after transformation of diatoms 27 clones were obtained in the condition corresponding to diatom transformed with TALE-Nuclease encoding plasmids (condition 1) and 17 in the condition corresponding to diatoms transformed with the empty vector (condition 2). 15 clones resulting from the condition 1 and 5 resulting from condition 2 were tested for targeted mutagenic events. For this purpose, genomic DNA was extracted and PCR surrounding the specific target sequence was performed. The presence of mutagenic events was measured by Deep sequencing analysis. Data are presented in the FIG. 6. Among all the tested clones, 3 presented a high rate of mutagenesis 1.5, 3.2 and 23.4% respectively. These three clones correspond to diatoms transformed with the TALE-Nuclease. While all other tested clones presented background levels of mutagenesis (<0.04%). Some examples of mutated sequences are presented in FIG. 7. One clone (n° 2) was further sub-cloned, and 7 sub-clones were analyzed. Among them, one presented 100% of mutated sequences.

Thus, TALE nuclease induces high frequency targeted mutagenesis (up to 23%). Moreover TALE-Nuclease induces mutagenesis on multiple copies of the YFP reporter gene stably integrated into the diatom genome.

### Example 5

### High Targeted Mutagenesis Frequency Induced Using TALE-Nuclease Targeting Endogenous Locus in the Diatom Thalassiosira pseudonana

To investigate the ability of a TALE-Nuclease to induce targeted mutagenesis in diatoms, one engineered TALE-Nuclease, called TP07 TALE-Nuclease encoded by the pCLS20885 (SEQ ID NO: 21) and pCLS20886 (SEQ ID NO: 22) plasmids designed to cleave the DNA sequence 5′ TGACTTTCCTCCCATGTTAGGTCCAGTGACAAGAAGGAATGAGGATGCA-3′ (SEQ ID NO: 23) within a gene encoding for the protein ID: 211853 were used. These TALE-Nuclease encoding plasmids were co-transformed with a plasmid conferring resistance to nourseothricin (NAT) in the diatom Thalassiosira pseudonana. The mutagenesis frequency induced by the designated TALE-Nuclease was measured by Deep sequencing on individual clones resulting from the transformations.

### Material and Methods

### Culture Conditions

Thalassiosira pseudonana clone CCMP1335 was grown in filtered Guillard's f/2 medium with silica [40°/°° w/v Sigma Sea Salts S9883, supplemented with 1× Guillard's f/2 marine water enrichment solution (Sigma G9903, 0.03°/°° w/v Na₂SiO₃.9H₂O)], in a Sanyo incubator (model MLR-351) at a constant temperature (20+/−0.5° C.). The incubator is equipped with white cold neon light tubes that produce an illumination of about 120 μmol photons m⁻² s⁻¹ and a photoperiod of 16 h light: 8 h darkness (illumination period from 9 AM to 1 AM). Liquid cultures were made in vented cap flasks put on an orbital shaker (Polymax 1040, Heidolph) with a rotation speed of 30 revolutions min⁻¹ and an angle of 5°.

### Genetic Transformation

10⁸ cells were collected from exponentially growing liquid cultures (concentration about 10⁶ cells/ml) by centrifugation (3000 rpm for 10 minutes at 20° C.). The supernatant was discarded and the cell pellet resuspended in 500 μl of fresh f/2 medium with silica. The cell suspension was then spread on the center one-third of a 10 cm 1% agar plate containing 40°/° ° sea salts supplemented with f/2 solution with silica. Two hours later, transformation was carried out using microparticle bombardment (Biolistic PDS-1000/He Particle Delivery System, BioRad). The protocol is adapted from Falciatore et al., (1999) and Apt et al., (1999) with minor modifications. Briefly, M17 tungsten particles (1.1 μm diameter, BioRad) were coated with 9 μg of a total amount of DNA composed of 3 μg of each monomer of TALE-Nucleases (pCLS20885 and pCLS20886) and 3 μg of the NAT (pCLS17714) (SEQ ID NO: 24) selection plasmid using 1.25M CaCl2 and 20 mM spermidin according to the manufacturer's instructions. As a negative control, beads were coated with a DNA mixture containing 3 μg of the NAT selection plasmid (pCLS17714) and 6 μg of an empty vector (pCLS0003) (SEQ ID NO: 4). Agar plates with the diatoms to be transformed were positioned at 7.5 cm from the stopping screen within the bombardment chamber (target shelf on position two). A burst pressure of 1550 psi and a vacuum of 20 Hg/in were used. Just after bombardment, cells were gently scrapped with 1 ml of f/2 medium supplemented with silica and directly seeded in vented cap flasks containing 100 ml of f/2 medium with silica. The resulting cell cultures were placed for 24 h in the incubator under a 16 h light: 8 h darkness cycle.

### Selection

One day post transformations, cells were counted and a volume of culture corresponding to 25.10⁶ cells was centrifugated at 3000 rpm for 10 min at 20° C. The cell pellet was resuspended in 1.5 ml of f/2 medium with silica and spread on five 10 cm 1% agar plates (40°/° ° sea salts supplemented with f/2 medium with silica) containing 200 μg ml⁻¹ nourseothricin (Werner Bioagents). Plates were then placed in the incubator under a 16 h light: 8 h darkness cycle for at least three weeks. 3 to 4 weeks after transformation, on average, resistant colonies resulting from a stable transformation were re-streaked on fresh 10 cm 1% agar plates containing 200 μg ml⁻¹ nourseothricin.

### Characterization

### Measure of the Mutagenesis Frequency by Deep Sequencing

Resistant colonies were picked and dissociated in 20 μl of lysis buffer (1% TritonX-100, 20 mM Tris-HCl pH8, 2 mM EDTA) in an eppendorf tube. Tubes were vortexed for at least 30 sec and then kept on ice for 15 min. After heating for 10 min at 85° C., tubes were cooled down at RT and briefly centrifuged to pellet cells debris. Supernatants were used immediately or stocked at 4° C. 5 μl of a 1:5 dilution in milliQ H2O of the supernatants, were used for each PCR reaction. The TP07 target was amplified using 1:5 dilution of the lysis colony, with specific primers flanked by specific adaptator needed for HTS sequencing on the 454 sequencing system (454 Life Sciences) using the primer TP07_For 5′-CCATCTCATCCCTGCGTGTCTCCGACTCAG-Tag-GGAAGTGAGTTGCAAACAC 3′ (SEQ ID NO: 25) and TP07 Rev 5′-CCTATCCCCTGTGTGCCTTGGCAGTCTCAG-CTTCAAGATGATATGAACTT-3′ (SEQ ID NO: 26). The PCR products were purified on magnetic beads (Agencourt AMPure XP, Beckman Coulter). 5000 to 10 000 sequences per sample were analyzed.

### Results

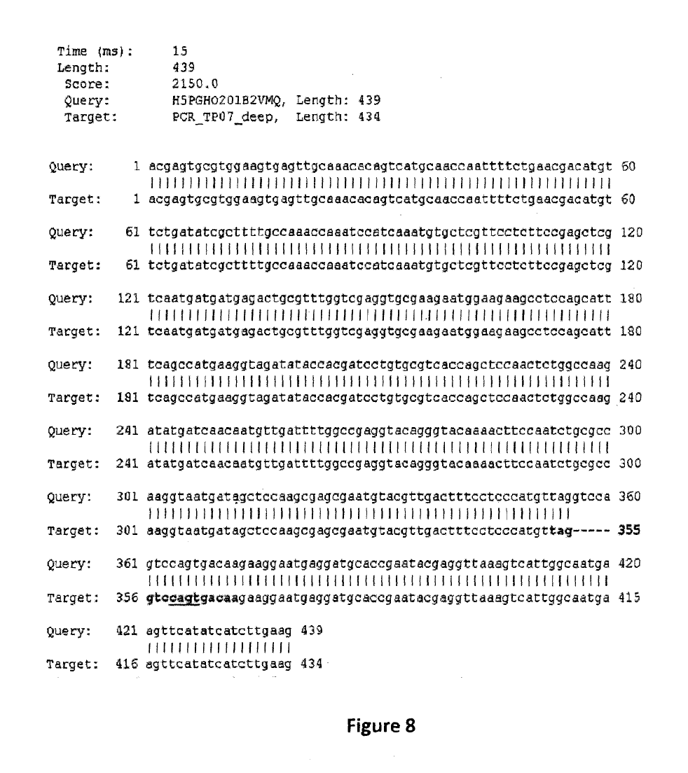

Three weeks after the plating of the transformed diatoms on the nourseothricin selective medium, one clone were obtained under the condition corresponding to the diatoms transformed with the TALE-Nuclease encoding plasmids (condition 1) and three under the condition corresponding to the diatoms transformed with the empty vector (condition 2). One clone resulting from the condition 1 and one resulting from condition 2 were tested for targeted mutagenic events. For this purpose, genomic DNA was extracted and PCR surrounding the specific target sequence was performed. The presence of mutagenic events was measured by Deep sequencing analysis. Among the tested clones, one presents a mutagenic event on 1,800 sequences analyzed (i.e. 0.05%). This clone corresponds to the diatoms transformed with the TALE-Nuclease. While all other tested clones present no mutagenic event. The mutated sequence identified is presented in FIG. 8.

Thus, TALE nuclease induces targeted mutagenesis at an endogenous locus (0.05%).

### Example 6

### High Targeted Mutagenesis Frequency Induced Using TALE-Nuclease (TP15) Targeting Endogenous Locus in the Diatom Thalassiosira pseudonana

To investigate the ability of a TALE-Nuclease to induce targeted mutagenesis in diatoms, one engineered TALE-Nuclease, called TP15_TALE-Nuclease encoded by the pCLS20726 (SEQ ID NO: 27) and pCLS20727 (SEQ ID NO: 28) plasmids designed to cleave the DNA sequence 5′-TTGGGTCTTGAAGGGATGTTGTCGGGAACCACGTTGGCCATGGAGTGGA-3′ (SEQ ID NO: 29) were used. These TALE-Nuclease encoding plasmids were co-transformed with a plasmid conferring resistance to nourseothricin (NAT) in the diatom Thalassiosira pseudonana. The mutagenesis frequency induced by the designated TALE-Nuclease was measured by Deep sequencing on individual clones resulting from the transformations.

### Materials and Methods

Thalassiosira pseudonana clone CCMP1335 was grown and transformed according to the method described in example 5 with M17 tungstene particles (1.1 μm diameter, BioRad) coated with 9 μg of a total amount of DNA composed of 3 μg of each monomer of TALE-Nucleases (pCLS20726 and pCLS20727) and 3 μg of the NAT (pCLS17714) (SEQ ID NO: 24) selection plasmid using 1.25M CaCl2 and 20 mM spermidin according to the manufacturer's instructions.

### Characterization

### Measure of the Mutagenesis Frequency by Deep Sequencing

After selection, resistant colonies were picked and dissociated according to the method described in example 5. Supernatants were used for each PCR reaction. The TP15 target was amplified using 1:5 dilution of the lysis colony, with specific primers flanked by specific adaptator needed for HTS sequencing on the 454 sequencing system (454 Life Sciences) using the primer TP15_For 5′-CCATCTCATCCCTGCGTGTCTCCGACTCAG-Tag-AATGCCCAAAGTATACACTGT-3′ (SEQ ID NO: 30) and TP15_Rev 5′ CCTATCCCCTGTGTGCCTTGGCAGTCTCAG-AATTCATTATCTCCGACTCTC-3′ (SEQ ID NO: 31). The PCR products were purified on magnetic beads (Agencourt AMPure XP, Beckman Coulter). 5000 to 10 000 sequences per sample were analyzed.

### Results

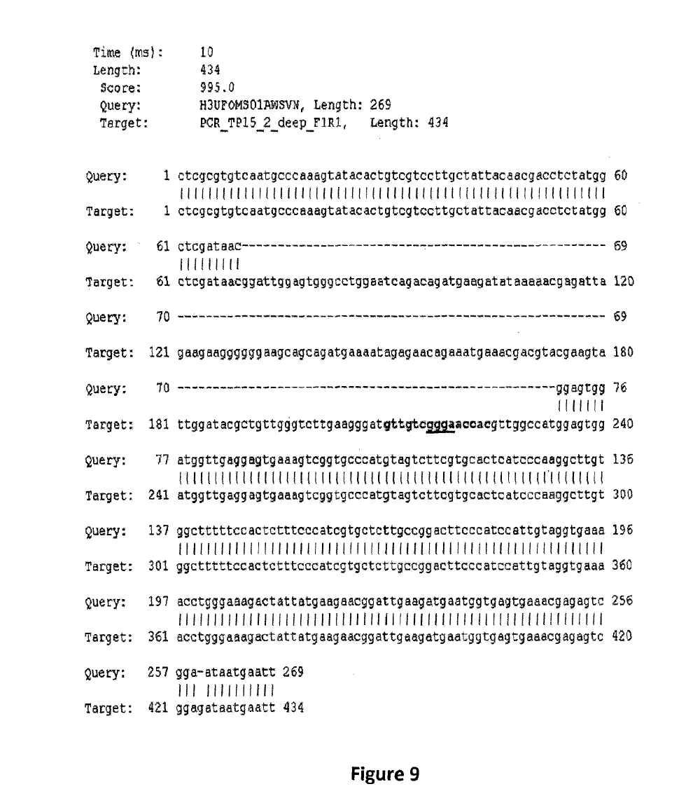

Three weeks after the plating of the transformed diatoms on the nourseothricin selective medium one clone was obtained under the condition corresponding to the diatoms transformed with the TALE-Nuclease encoding plasmids (condition 1) and one under the condition corresponding to the diatoms transformed with the empty vector (condition 2). One clone resulting from the condition 1 and one resulting from the condition 2 were tested for targeted mutagenic events. For this purpose, genomic DNA was extracted and PCR surrounding the specific target sequence was performed. The presence of mutagenic events was measured by Deep sequencing analysis. Among the tested clones, one presents a mutagenic event on 7,192 sequences analyzed (i.e. 0.014%). This clone corresponds to diatoms transformed with the TALE-Nuclease. While all other tested clones present no mutagenic event. The mutated sequence identified is presented in FIG. 9.

Thus, TALE nuclease induces targeted mutagenesis at an endogenous locus (0.014%).

### Example 7

### Gene Targeting Induced by an Engineered Meganuclease (PTRI02) in Phaeodactylum tricornutum

To investigate the ability of a rare-cutting endonuclease to induce gene targeting frequency into diatoms, one engineered meganuclease, called PTRI02 encoded by the pCLS17181 (SEQ ID NO: 12) plasmids designed to cleave the DNA sequence 5′ TTTTGACGTCGTACGGTGTCTCCG-3′ (SEQ ID NO: 13) was used. This meganuclease was co-transformed with a plasmid conferring resistance to nourseothricin (NAT) and a DNA matrix plasmid pCLS19635 (SEQ ID NO: 32) composed of two arms homologous to the targeted sequence separated by a heterologous fragment, in a wild type diatom strain. The individual clones resulting from the transformation were screened by PCR for the presence of gene targeting events and the homologous recombination frequency was measured by Deep sequencing.

### Materials and Methods

Phaeodactylum tricornutum Bohlin clone CCMP2561 was grown and transformed according to the methods described in example 1 with M17 tungstene particles (1.1 μm diameter, BioRad) coated with 9 μg of a total amount of DNA composed of 3 μg of meganuclease pCLS17181 (SEQ ID NO: 12), 3 μg of the NAT selection plasmid (pCLS16604) (SEQ ID NO: 3) and 3 μg of the DNA matrix plasmid (pCLS19635) (SEQ ID NO: 32) using 1.25M CaCl2 and 20 mM spermidin according to the manufacturer's instructions. As negative control, beads were coated with a DNA mixture containing 3 μg of the NAT selection plasmid (pCLS16604), 3 μg of the DNA matrix plasmid (pCLS19635) (SEQ ID NO: 32) and 3 μg of an empty vector (pCLS0003) (SEQ ID NO: 4).

### Characterization

### A-Colony Screening

After selection, resistant colonies were picked and dissociated according to the methods of example 1. Supernatants were used for each PCR reaction. Specific primers for meganuclease screen: Meganuclease_For 5′-TTAACAATTGAATCTCGCCTATTCATGGTG-3′ (SEQ ID NO: 8) and Meganuclease_Rev 5′-TAGCGCTCGAGTTACTAAGGAGAGGACTTTTTCTT-3′ (SEQ ID NO: 9).

### B-Identification of Homologous Gene Targeting Event

The detection of targeted integration is performed by specific PCR amplification using a primer located within the heterologous insert of the DNA repair matrix and one located on genomic sequence outside of the homology arm. 1/20 of the lysis colony was used for PCR screening.

For the screen left, PTRI02_HGT_Left_For (located outside of the homology): 5′-CCGGCCAGAGTCGAATTGGCCACGTGG-3′ (SEQ ID NO: 33) and Insert_HGT_Left_Rev (located in the heterologous insert): 5′-AATTGCGGCCGCGGTCCGGCGC-3′ (SEQ ID NO: 34). For the screen right, PTRI02_HGT_Right_For (located in the heterologous insert): 5′-TTAAGGCGCGCCGGACCGCGGC-3′ (SEQ ID NO: 35) and PTRI02_HGT_Right_Rev (located outside of the homology): 5′-GACGACGACGAAAACGTCTTGCGTCCG-3′ (SEQ ID NO: 36).

### C-Measure of the Homologous Gene Targeting Frequency by Deep Sequencing

In order to measure the homologous recombination frequency induced by the PTRI02 meganuclease, two successive PCR were performed. The first PCR (locus specific) was performed using the primers PTRI02_HGT_Left_For: 5′-CCGGCCAGAGTCGAATTGGCCACGTGG-3′(SEQ ID NO: 33) and PTRI02_HGT_Right_Rev: 5′-GACGACGACGAAAACGTCTTGCGTCCG-3′ (SEQ ID NO: 36). The PCR product was then purified on gel and an aliquot ( 1/60 of the elution) was used for the nested PCR using the primers

PTRI02_For
(SEQ ID NO: 14)
5′-
CCATCTCATCCCTGCGTGTCTCCGACTCAG-TAG-
TCAGCTCCATTGGAATGTTGGC -3′
and
PTRI02_Rev
(SEQ ID NO: 15)
5′-
CCTATCCCCTGTGTGCCTTGGCAGTCTCAG-
CCCTCCGACCAGGGAACTTACTC -3′.

PTRI02_For and PTRI02_Rev2are flanked by specific adaptator needed for HTS sequencing on the 454 sequencing system (454 Life Sciences). The PCR products were purified on magnetic beads (Agencourt AMPure XP, Beckman Coulter). 5000 to 10 000 sequences per sample were analyzed.

### Results

Three weeks after the transformation of the diatoms, 23 clones were obtained in the condition corresponding to the transformation performed with the meganuclease PTRI02 and the DNA matrix encoding plasmids (condition 1). Among them, 8/28 (i.e. 28.5%) were positive for both the presence of meganuclease encoding plasmid and HGT events. Finally, 21 clones resulting from the transformation with the DNA matrix and the empty vector were obtained (condition 2). None of them were positive for the presence of HGT events.

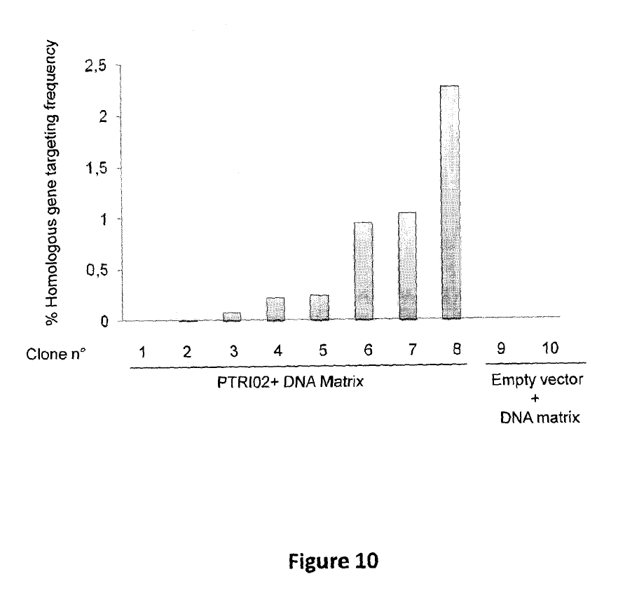

The homologous gene targeting frequency was determined by Deep sequencing on the 8 clones positive for HGT events and 2 clones from condition 2 negative for HGT, used here as negative control. Whereas the samples corresponding to the 8 positive clones (condition 1) present 0; 0.01, 0.079; 0.213; 0.238; 0.949; 1.042; 2.277 of HGT positive PCR fragments, this percentage is zero in the 2 samples corresponding to the condition 2, negative for HGT event screening (FIG. 10).

To conclude, the use of one meganuclease able to cleave an endogenous target in combination with a DNA matrix homologous to the targeted sequence allows homologous gene targeting events in diatoms (up to 2%).

### Example 8

### Gene Targeting Induced by an Engineered Meganuclease (PTRI20) in Phaeodactylum tricornutum

To investigate the ability of a rare-cutting endonuclease to induce gene targeting frequency into diatoms, one engineered meganuclease, called PTRI20 encoded by the pCLS17038 (SEQ ID NO: 1) plasmids designed to cleave the DNA sequence 5′ GTTTTACGTTGTACGACGTCTAGC-3′ (SEQ ID NO: 2) was used. This meganuclease was co-transformed with a plasmid conferring resistance to nourseothricin (NAT) and a DNA matrix plasmid pCLS19773 (SEQ ID NO: 37) composed of two arms homologous to the targeted sequence separated by a heterologous fragment, in a wild type diatom strain. The individual clones resulting from the transformation were screened by PCR for the presence of gene targeting events and the homologous recombination frequency was measured by Deep sequencing.

### Materials and Methods

Phaeodactylum tricornutum Bohlin clone CCMP2561 was grown CCMP2561 was grown and transformed according to the methods described in example 1 with M17 tungstene particles (1.1 μm diameter, BioRad) coated with 9 μg of a total amount of DNA composed of 3 μg of meganuclease pCLS17038 (SEQ ID NO: 1), 3 μg of the NAT selection plasmid (pCLS16604) (SEQ ID NO: 3) and 3 μg of the DNA matrix plasmid (pCLS19773) (SEQ ID NO: 37) using 1.25M CaCl2 and 20 mM spermidin according to the manufacturer's instructions. As negative control, beads were coated with a DNA mixture containing 3 μg of the NAT selection plasmid (pCLS16604), 3 μg of the DNA matrix plasmid (pCLS19773) (SEQ ID NO: 37) and 3 μg of an empty vector (pCLS0003) (SEQ ID NO: 4).

### Characterization

### A-Colony Screening

After selection, resistant colonies were picked and dissociated according to the methods of example 1. Supernatants were used for each PCR reaction. Specific primers for meganuclease screen: Meganuclease_For 5′-TTAACAATTGAATCTCGCCTATTCATGGTG-3′ (SEQ ID NO: 8) and Meganuclease_Rev 5′-TAG CGCTCGAGTTACTAAGGAGAGGACTTTTTCTT-3′ (SEQ ID NO: 9).

### B-Identification of Homologous Gene Targeting Event

The detection of targeted integration is performed by specific PCR amplification using a primer located within the heterologous insert of the DNA repair matrix and one located on genomic sequence outside of the homology arm. 1/20 of the lysis colony was used for PCR screening.

For the screen left, PTRI20_HGT_Left_For (located outside of the homology): 5′-GCAGCGTACGCAGCCATAGTCCGGAACG-3′ (SEQ ID NO: 38) and Insert_HGT_Left_Rev (located in the heterologous insert): 5′-AATTGCGGCCGCGGTCCGGCGC-3′ (SEQ ID NO: 34). For the screen right, PTRI20_HGT_Right_For (located in the heterologous insert): 5′-TGTTTTACGTTGTTTAAGGCGCGCCG-3′ (SEQ ID NO: 39) and PTRI20_HGT_Right_Rev (located outside of the homology): 5′-CCGCATCTCAATCACGTCTTGTTGAAGC-3′ (SEQ ID NO: 40).

### C-Measure of the Homologous Gene Targeting Frequency by Deep Sequencing

In order to measure the homologous recombination frequency induced by the PTRI20 meganuclease, two successive PCR were performed. The first PCR (locus specific) was performed using the primers PTRI20_HGT_Left_For: 5′-GCAGCGTACGCAGCCATAGTCCGGAACG-3′ (SEQ ID NO: 38) and PTRI20_HGT_Right_Rev: 5′-CCGCATCTCAATCACGTCTTGTTGAAGC-3′ (SEQ ID NO: 40). The PCR product was then purified on gel and an aliquot ( 1/60 of the elution) was used for the nested PCR using the primers

PTRI20_For
(SEQ ID NO: 5)
5′-
CGGTTGTCATGGATAGCGGAGC-TAG-
TCAGCTCCATTGGAATGTTGGC -3′
and
PTRI20_Rev
(SEQ ID NO: 6)
5′- CCCCAGACGATTCGAAGTCGTCC-
CCCTCCGACCAGGGAACTTACTC -3′.

PTRI20_For and PTRI20_Rev are flanked by specific adaptator needed for HTS sequencing on the 454 sequencing system (454 Life Sciences). The PCR products were purified on magnetic beads (Agencourt AMPure XP, Beckman Coulter). 5000 to 10 000 sequences per sample were analyzed.

### Results

Three weeks after the transformation of the diatoms, 11 clones were obtained in the condition corresponding to the transformation performed with the meganuclease PTRI20 and the DNA matrix encoding plasmids (condition 1). Among them, 9 were screened for the presence of the meganuclease encoding plasmid and HGT events and 3 were positive for both (i.e. 33%). Finally, 16 clones resulting from the transformation with the DNA matrix and the empty vector were obtained (condition 2). Among them, 12 were tested for the presence of HGT events and none of them were positive for HGT event.

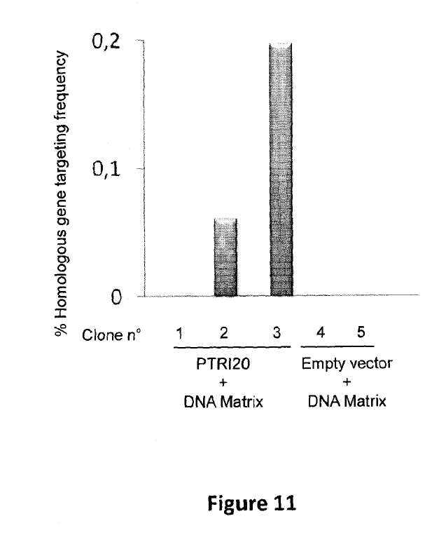

The homologous gene targeting frequency was determined by Deep sequencing on the 3 clones positive for HGT events and 2 clones from condition 2 negative for HGT, used here as negative control. Whereas the samples corresponding to the 3 positive clones (condition 1) present 0; 0.06 and 0.197% of HGT positive PCR fragments, this percentage is zero in the 2 samples corresponding to the condition 2, negative for HGT event screening (FIG. 11).

To conclude, the use of one meganuclease able to cleave an endogenous target in combination with a DNA matrix homologous to the targeted sequence allows homologous gene targeting events in diatoms (up to 0.19%).

### Example 9

### Targeted Mutagenesis Induced by a TALE-Nuclease Targeting UDP-Glucose Pyrophosporylase (UGPase) Gene

In order to determine the ability of a TALE-Nuclease to induce targeted mutagenesis in UGPase gene (SEQ ID NO: 41) in diatoms, one engineered TALE-Nuclease, called UGP TALE-Nuclease encoded by the pCLS19745 (SEQ ID NO: 42) and pCLS19749 (SEQ ID NO: 43) plasmids designed to cleave the DNA sequence 5′ TGCCGCCTTCGAGTCGACCTATGGTAGTCTCGTCTCGGGTGATTCCGGAA-3′ (SEQ ID NO: 44) were used. These TALE-Nuclease encoding plasmids were co-transformed with a plasmid conferring resistance to nourseothricin (NAT) in a wild type diatom strain. The individual clones resulting from the transformation were screened for the presence of mutagenic events which lead to UGPase gene inactivation.

### Materials and Methods

Phaeodactylum tricornutum Bohlin clone CCMP2561 was grown and transformed according to the method described in example 1 with M17 tungstene particles (1.1 μm diameter, BioRad) coated with 9 μg of a total amount of DNA composed of 1.5 μg (experiment 2) or 3 μg (experiment 1) of each monomer of TALE-Nucleases (pCLS19745 and pCLS19749), 3 μg of the NAT selection plasmid (pCLS16604) (SEQ ID NO: 3) and 3 μg of an empty vector (pCLS0003) (SEQ ID NO: 4) using 1.25M CaCl2 and 20 mM spermidin according to the manufacturer's instructions. As a negative control, beads were coated with a DNA mixture containing 3 μg of the NAT selection plasmid (pCLS16604) and 6 μg of an empty vector (pCLS0003) (SEQ ID NO: 4).

### Characterization

### A-Colony Screening

After selection, resistant colonies were picked and dissociated according to method described in example 1. Supernatants were used for each PCR reaction. Specific primers for TALE-Nuclease screens: TALE-Nuclease_For 5′-AATCTCGCCTATTCATGGTG-3′ (SEQ ID NO: 49) and HA_Rev 5′-TAATCTGGAACATCGTATGGG-3′ (SEQ ID NO: 50) and TALE-Nuclease_For 5′-AATCTCGCCTATTCATGGTG-3′ (SEQ ID NO: 49) and STag_Rev 5′-TGTCTCTCGAACTTGGCAGCG-3′ (SEQ ID NO: 51).

### B-Identification of Mutagenic Events

The UGPase target was amplified using a 1:5 dilution of the colony lysates with sequence specific primers flanked by adaptators needed for HTS sequencing on a 454 sequencing system (454 Life Sciences) and the two following primers: UGP_For 5′-CCATCTCATCCCTGCGTGTCTCCGACTCAG-Tag-GTTGAATCGGAATCGCTAACTCG-3′ (SEQ ID NO: 45) and UGP_Rev 5′-CCTATCCCCTGTGTGCCTTGGCAGTCTCAG GACTTGTTTGGCGGTCAAATCC-3′ (SEQ ID NO: 46).

The PCR products were purified on magnetic beads (Agencourt AMPure XP, Beckman Coulter) and quantified with a NanoDrop 1000 spectrophotometer (Thermo Scientifioc). 50 ng of the amplicons were denatured and then annealed in 10 μl of the annealing buffer (10 mM Tris-HCl pH8, 100 mM NaCl, 1 mM EDTA) using an Eppendorf MasterCycle gradient PCR machine. The annealing program is as follows: 95° C. for 10 min; fast cooling to 85° C. at 3° C./sec; and slow cooling to 25° C. at 0.3° C./sec. The totality of the annealed DNA was digested for 15 min at 37° C. with 0.5 μl of the T7 Endonuclease I (10 U/μl) (M0302, Biolabs) in a final volume of 20 μl (1×NEB buffer 2, Biolabs). 10 μl of the digestion were then loaded on a 10% polyacrylamide MiniProtean TBE precast gel (BioRad). After migration the gel was stained with SYBRgreen and scanned on a Gel Doc XR+ apparatus (BioRad).

### C-Measure of the Mutagenesis Frequency by Deep Sequencing

The UGPase target was amplified with specific primers flanked by adaptators needed for HTS sequencing on the 454 sequencing system (454 Life Sciences) using the primer UGP_For 5′-GTTGAATCGGAATCGCTAACTCG-3′ (SEQ ID NO: 47) and UGP_Rev 5′-GACTTGTTTGGCGGTCAAATCC-3′ (SEQ ID NO: 48). 5000 to 10 000 sequences per sample were analyzed.

### D-Phenotypic Characterization of UDP KO Clones by Bodipy Labeling

Cells were re-suspended at the density of 5.10⁵ cells/ml and washed twice in culture medium (filtered Guillard's f/2 medium without silica). The bodipy labeling was performed with 10 μM of final concentration of Bodipy 493/503 (Molecular Probe) in presence of 10% of DMSO during 10 minutes at room temperature in the dark. The fluorescence intensity was measured by flow cytometry at 488 nM (MACSQuant Analyzer, Miltenyi Biotec).

### Results

Three independent experiments were performed using the TALE-Nuclease targeting the UGPase gene. For each of them, the presence of mutagenic events in the clones obtained three weeks after diatoms transformation was analyzed.

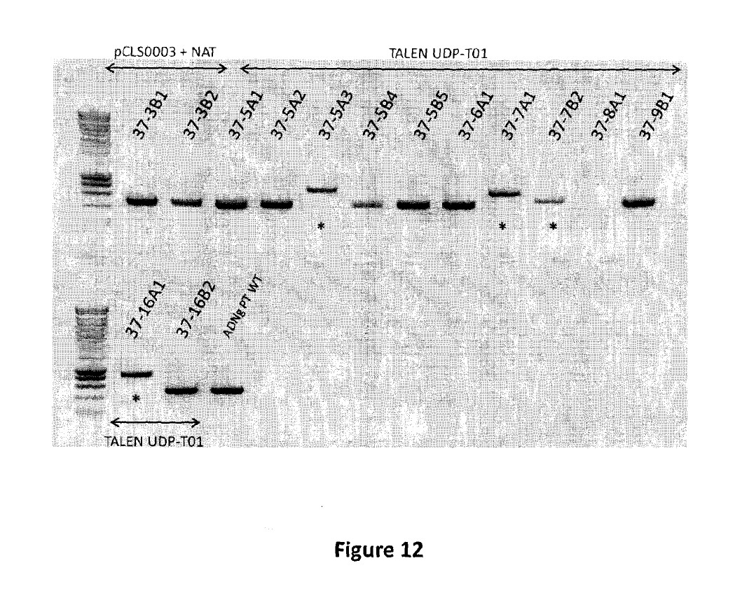

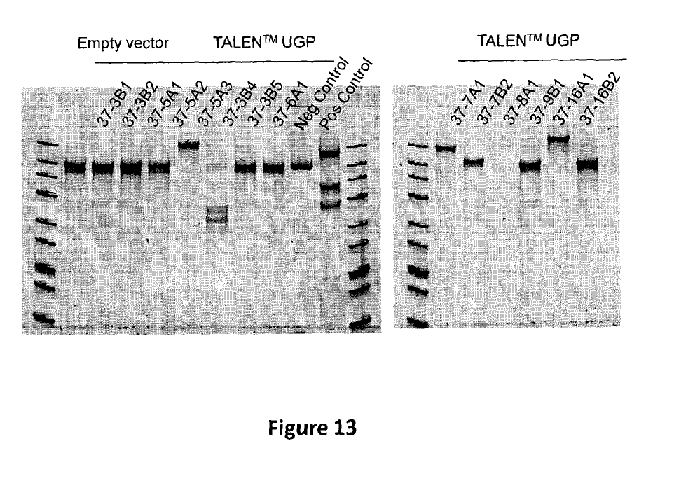

For the first experiment, 18 clones were obtained in the condition corresponding to diatoms transformed with TALE-Nuclease encoding plasmids (condition 1). Finally, 6 clones resulting from the transformation with the empty vector were obtained (condition 2). The UGPase target amplification was performed on 12 clones obtained in the condition 1 and 2 clones obtained in the condition 2. On the 12 clones tested, 4 present a PCR band higher than expected showing a clear mutagenic event, 1 presents no amplification of the UGPase target, 7 present a band at the wild type size. A T7 assay was assessed on these 12 clones (FIG. 12). One clone among them was positive in T7 assay which reflects the presence of mutagenic events (FIG. 13). As expected no signal was detected in the 2 clones from the condition corresponding to empty vector (condition 2).

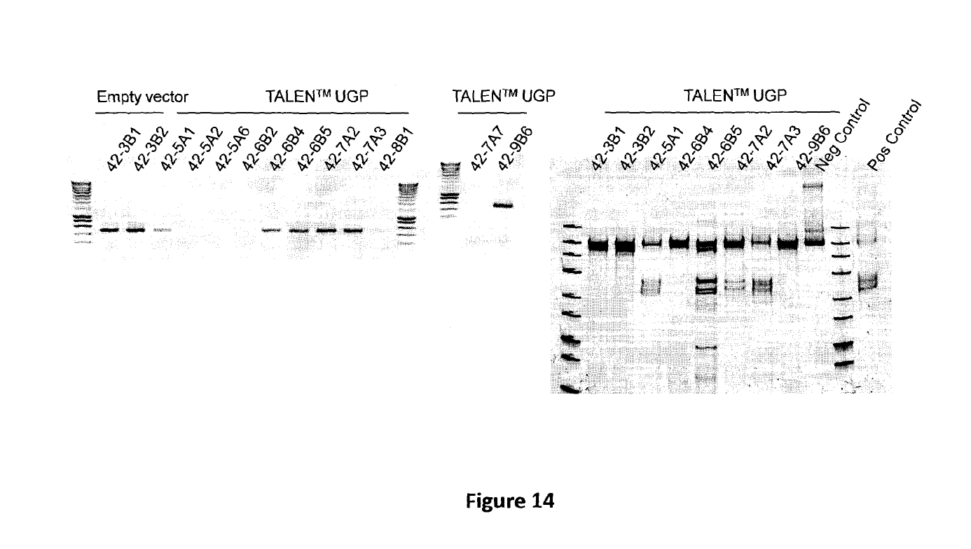

For the second experiment, 62 clones were obtained in the condition corresponding to diatoms transformed with TALE-Nuclease encoding plasmids (condition 1). Among them, 36 were tested for the presence of the DNA sequences encoding both TALE-Nuclease monomers. 11/36 (i.e. 30.5%) were positive for both TALE-Nuclease monomers DNA sequences. Finally, 38 clones resulting from the transformation with the empty vector were obtained (condition 2). The UGPase target amplification was performed on 11 clones obtained in the condition 1 and 2 clones obtained in the condition 2. On the 11 clones tested, 5 present no amplification of the UGPase target, 6 present a band at the wild type size (FIG. 14).

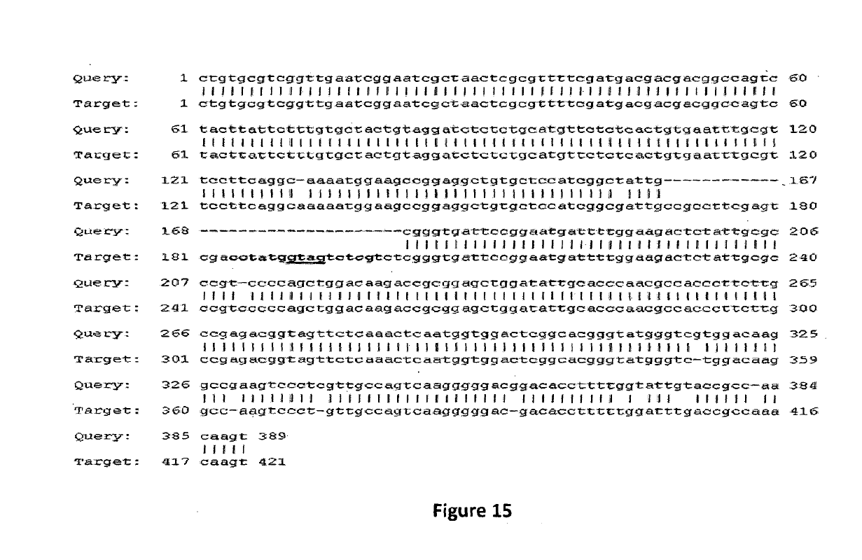

In order to identify the nature of the mutagenic event in the 4 clones displaying a higher PCR amplification product from experiment 1 (FIG. 12), we sequenced these fragments. All of them present an insertion of 261 bp (37-5A3), 228 bp (37-7A1), 55 bp (37-7B2) and 330 bp (37-16A1), respectively leading to the presence of stop codon in the coding sequence. The clone 37-3B4 presenting a positive signal for T7 assay was characterized by Deep sequencing. The mutagenesis frequency in this clone was 86% with several type of mutagenic event (either insertion or deletion). An example of mutated sequences is presented in FIG. 15.

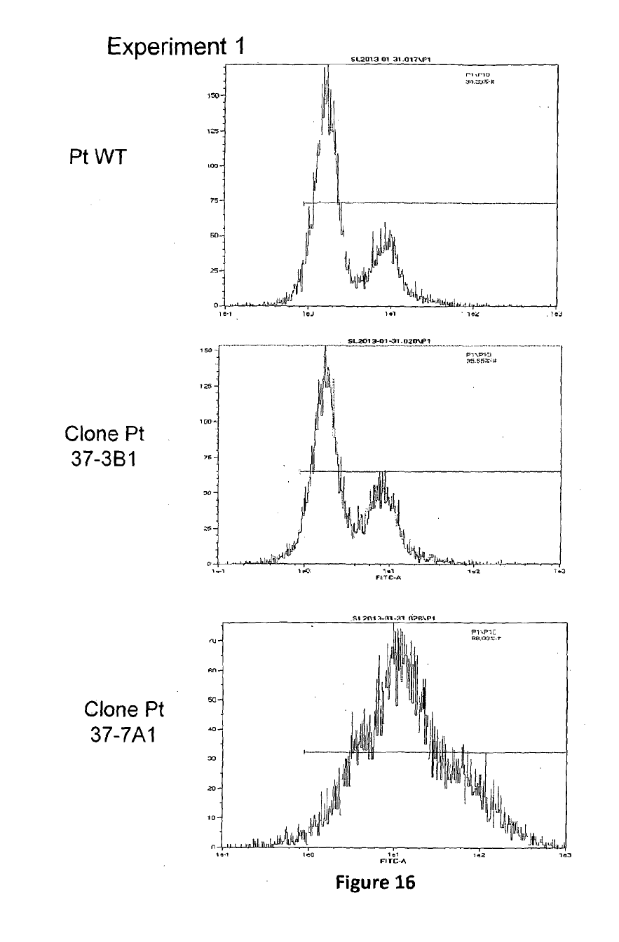

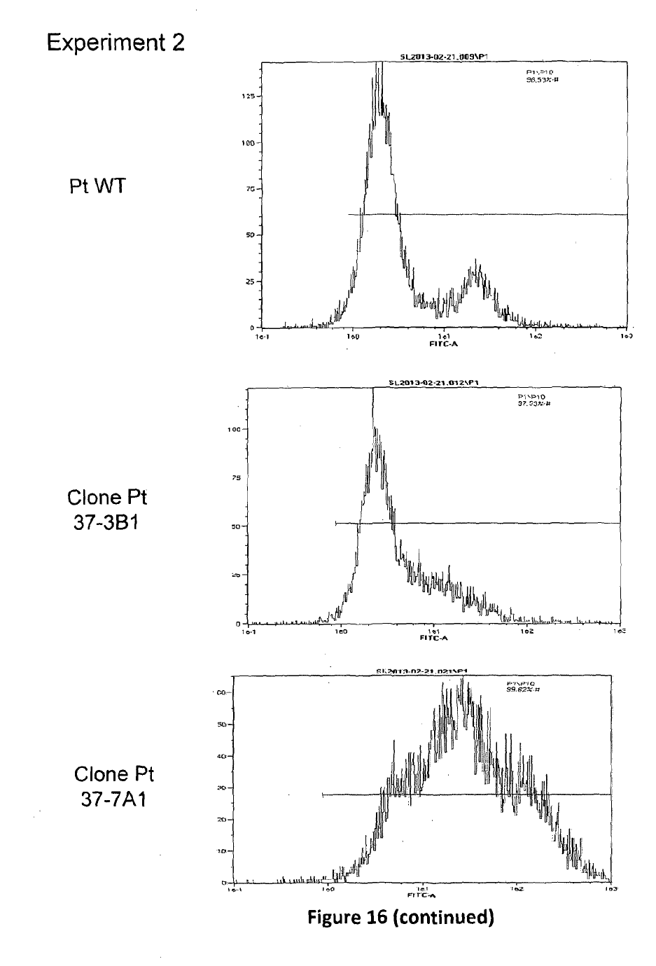

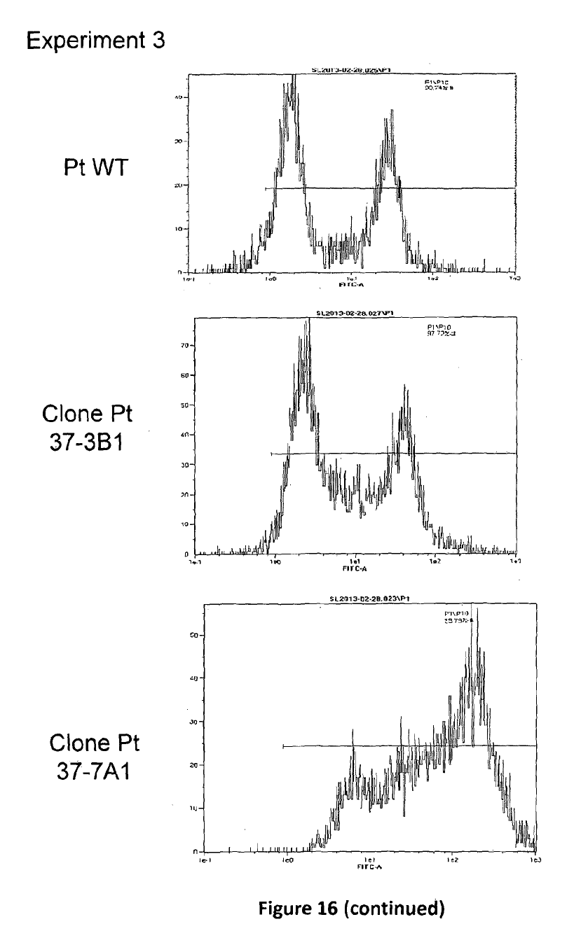

To investigate the impact of UGPase gene inactivation on lipid content, a Bodipy 493/503 labeling (Molecular Probe) was performed on one clone harboring a mutagenic event in the UGPase target (37-7A1 CCAP 1055/12). In parallel, the Phaeodactylum tricornutum wild type strain and one clone resulting from the transformation with the empty vector were tested. The results are presented in FIG. 16. We observed an increase of the fluorescence intensity in the clone presenting an inactivation of the UGPase gene compared to the two control strains. This experiment was reproduced 3 times and a shift in the fluorescence intensity was observed at each time. As Bodipy labeling reflects the lipid content of the cells, these results demonstrated a robust and reproducible increase of the lipid content of the mutated strains.

Thus, a TALE nuclease targeting the UGPase gene induces a reproducible (2 independent experiments), and at high frequency, targeted mutagenesis (up to 100%). Moreover, the inactivation of the UGPase gene leads to a strong and reproducible increase of lipid content in bodipy labeling.

### Example 10

### Targeted Mutagenesis Induced by a TALE-Nuclease Targeting a Putative Elongase Gene

In order to investigate the ability of a TALE-Nuclease to induce targeted mutagenesis in the putative elongase gene (SEQ ID NO: 52) in diatoms, one engineered TALE-Nuclease, called elongase_TALE-Nuclease encoded by the pCLS19746 (SEQ ID NO: 53) and pCLS19750 (SEQ ID NO: 54) plasmids designed to cleave the DNA sequence 5′ TCTTTTCCCTCGTCGGCatgctccggacctttCCCCAGCTTGTACACAA-3′ (SEQ ID NO: 55) was used. Although this TALE-nuclease targets a sequence coding a protein with unknown function, this target present 86% of sequence identity with the mRNA of the fatty acid elongase 6 (ELOVL6) in Taeniopygia guttata, and 86% of sequence identity with the elongation of very long chain fatty acids protein 6-like (LOC100542840) in meleagris gallopavo.

These TALE-Nuclease encoding plasmids were co-transformed with a plasmid conferring resistance to nourseothricin (NAT) in a wild type diatom strain. The individual clones resulting from the transformation were screened for the presence of mutagenic events which lead to elongase gene inactivation.

### Materials and Methods

Phaeodactylum tricornutum Bohlin clone CCMP2561 was grown and transformed according to the methods described in example 1 with M17 tungstene particles (1.1 μm diameter, BioRad) coated with 9 μg of a total amount of DNA composed of 1.5 μg of each monomer of TALE-Nucleases (pCLS19746 (SEQ ID NO: 53) and pCLS19750 (SEQ ID NO: 54)), 3 μg of the NAT selection plasmid (pCLS16604) (SEQ ID NO: 3) and 3 μg of an empty vector (pCLS0003) (SEQ ID NO: 4) using 1.25M CaCl2 and 20 mM spermidin according to the manufacturer's instructions.

### Characterization

### A-Colony Screening

After selection, resistant colonies were picked and dissociated according to the method described in example 1. Supernatants were used were used for each PCR reaction. Specific primers for TALE-Nuclease screens: TALE-Nuclease_For 5′-AATCTCGCCTATTCATGGTG-3′ (SEQ ID NO: 49) and HA_Rev 5′-TAATCTGGAACATCGTATGGG-3′ (SEQ ID NO: 50). TALE-Nuclease_For 5′-AATCTCGCCTATTCATGGTG-3′ (SEQ ID NO: 49) and S-Tag_Rev 5′-TGTCTCTCGAACTTGGCAGCG-3′ (SEQ ID NO: 51).

### B-Identification of Mutagenic Event

The elongase target was amplified using a 1:5 dilution of the lysis colony with sequence specific primers flanked by adaptators needed for HTS sequencing on the 454 sequencing system (454 Life Sciences) and the two following primers: elongase_For 5′-CCATCTCATCCCTGCGTGTCTCCGACTCAG-Tag-AAGCGCATCCGTTGGTTCC-3′ (SEQ ID NO: 56) and elongase_Rev 5′-CCTATCCCCTGTGTGCCTTGGCAGTCTCAG TCAATGAGTTCACTGGAAAGGG-3′ (SEQ ID NO: 57).

The PCR products were purified on magnetic beads (Agencourt AMPure XP, Beckman Coulter) and quantified with a NanoDrop 1000 spectrophotometer (Thermo Scientifioc). 50 ng of the amplicons were denatured and then annealed in 10 μl of annealing buffer (10 mM Tris-HCl pH8, 100 mM NaCl, 1 mM EDTA) using an Eppendorf MasterCycle gradient PCR machine. The annealing program is as follows: 95° C. for 10 min; fast cooling to 85° C. at 3° C./sec; and slow cooling to 25° C. at 0.3° C./sec. The totality of the annealed DNA was digested for 15 min at 37° C. with 0.5 μl of the T7 Endonuclease I (10 U/μl) (M0302 Biolabs) in a final volume of 20 μl (1×NEB buffer 2, Biolabs). 10 μl of the digestion were then loaded on a 10% polyacrylamide MiniProtean TBE precast gel (BioRad). After migration the gel was stained with SYBRgreen and scanned on a Gel Doc XR+ apparatus (BioRad).

### C-Measure of the Mutagenesis Frequency by Deep Sequencing

The elongase target was amplified with sequence specific primers flanked by adaptators needed for HTS sequencing on the 454 sequencing system (454 Life Sciences) using the primer elongase_For 5′-AAGCGCATCCGTTGGTTCC-3′ (SEQ ID NO: 58) and Delta 6 elongase_Rev 5′-TCAATGAGTTCACTGGAAAGGG-3′ (SEQ ID NO: 59). 5000 to 10 000 sequences per sample were analyzed.

### Results

Three weeks after the transformation of the diatoms, 62 clones were obtained in the condition corresponding to the transformation performed with the TALE-Nuclease encoding plasmids (condition 1). Among them, 35 were tested for the presence of both TALE-Nuclease monomers DNA sequences. 11/27 (i.e. 40.7%) were positive for both TALE-Nuclease monomers DNA sequences. Finally, 38 clones resulting from the transformation with the empty vector were obtained (condition 2).

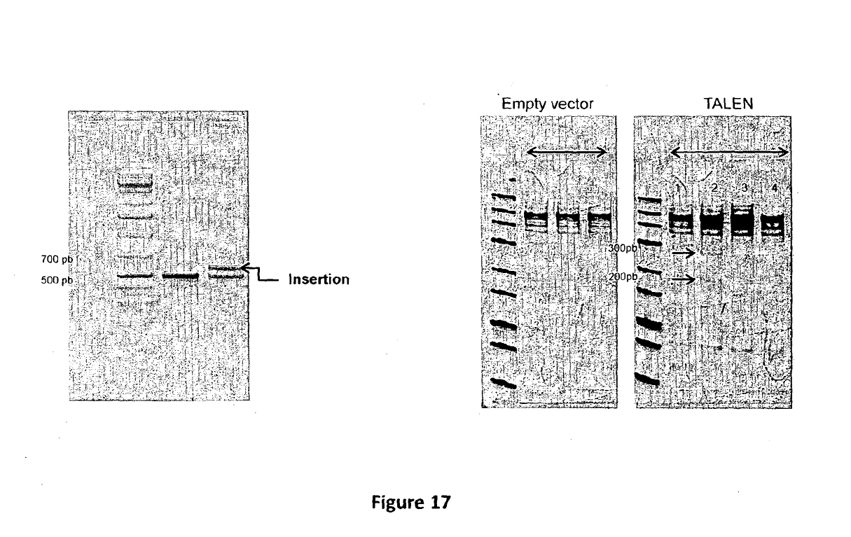

The 11 clones, positive for both TALE-Nuclease monomers DNA sequences were tested with the T7 assay. The Phaeodactylum tricornutum strain, as well as four clones resulting from the transformation with the empty vector, were tested in parallel. Four clones presented no amplification. Because the amplification of another locus is possible, the quality of the lysates is not questioned. So the absence of amplification could suggest the presence of a large mutagenic event at the elongase locus. One clone showed in equal proportions a PCR product at the expected size and another one with a higher weight, actually demonstrating a clear mutagenic event (FIG. 17). One clone was positive in the T7 assay, which reflects the presence of mutagenic events and 9 clones presented no signal in the T7 assay. As expected no signal was detected in the condition corresponding to the empty vector or the Phaeodactylum tricornutum wild type strain.

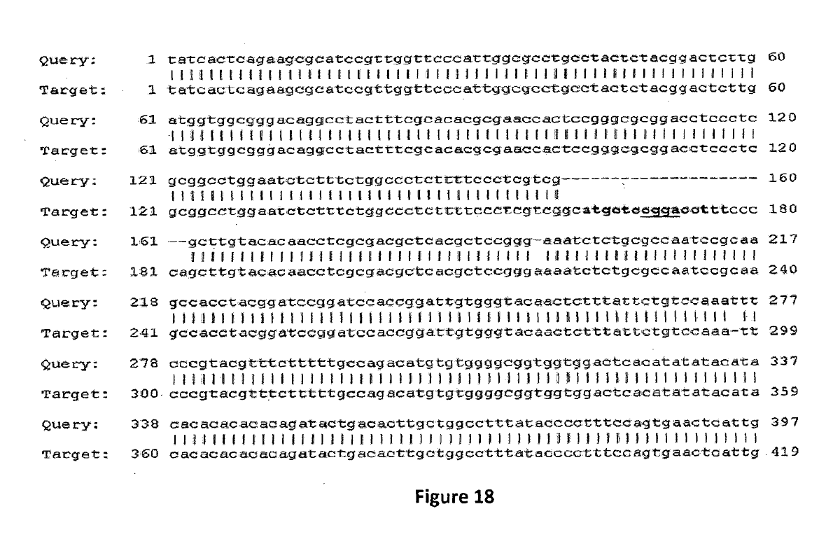

In order to identify the nature of the mutagenic event in the clone displaying a higher PCR amplification product, we sequenced this fragment. An insertion of 83 bp was detected leading to presence of stop codon in the coding sequence. The clone presenting a positive T7 signal was characterized by Deep sequencing. The mutagenesis frequency in this clone was 5.9% with one type of mutation (deletion of 22 bp). An example of mutated sequences is presented in FIG. 18.

### REFERENCES

Apt, K. E., P. G. Kroth-Pancic, et al. (1996). “Stable nuclear transformation of the diatom Phaeodactylum tricornutum.” Mol Gen Genet 252(5): 572-9.
Arimondo, P. B., C. J. Thomas, et al. (2006). “Exploring the cellular activity of camptothecin-triple-helix-forming oligonucleotide conjugates.” Mol Cell Biol 26(1): 324-33.
Armbrust, E. V., J. A. Berges, et al. (2004). “The genome of the diatom Thalassiosira pseudonana: ecology, evolution, and metabolism.” Science 306(5693): 79-86.
Arnould, S., C. Delenda, et al. (2011). “The I-Crel meganuclease and its engineered derivatives: applications from cell modification to gene therapy.” Protein Eng Des Sel 24(1-2): 27-31.
Baulcombe, D. (2004). “RNA silencing in plants.” Nature 431(7006): 356-63.
Boch, J., H. Scholze, et al. (2009). “Breaking the code of DNA binding specificity of TAL-type III effectors.” Science 326(5959): 1509-12.
Bowler, C., A. E. Allen, et al. (2008). “The Phaeodactylum genome reveals the evolutionary history of diatom genomes.” Nature 456(7219): 239-44.
Cannata, F., E. Brunet, et al. (2008). “Triplex-forming oligonucleotide-orthophenanthroline conjugates for efficient targeted genome modification.” Proc Natl Acad Sci USA 105(28): 9576-81.
Christian, M., T. Cermak, et al. (2010). “Targeting DNA double-strand breaks with TAL effector nucleases.” Genetics 186(2): 757-61.
Critchlow, S. E. and S. P. Jackson (1998). “DNA end-joining: from yeast to man.” Trends Biochem Sci 23(10): 394-8.
Depicker, A. and M. V. Montagu (1997). “Post-transcriptional gene silencing in plants.” Curr Opin Cell Biol 9(3): 373-82.
Dunahay, T. G., E. E. Jarvis, et al. (1995). “Genetic transformation of the diatoms Cyclotella Cryptica and Navicula Saprophila.” Journal of Phycology 31(6): 1004-1012.
Eisenschmidt, K., T. Lanio, et al. (2005). “Developing a programmed restriction endonuclease for highly specific DNA cleavage.” Nucleic Acids Res 33(22): 7039-47.
Falciatore, A., R. Casotti, et al. (1999). “Transformation of Nonselectable Reporter Genes in Marine Diatoms.” Mar Biotechnol (NY) 1(3): 239-251.
Galetto, R., P. Duchateau, et al. (2009). “Targeted approaches for gene therapy and the emergence of engineered meganucleases.” Expert Opin Biol Ther 9(10): 1289-303.
Gruber, A., S. Vugrinec, et al. (2007). “Protein targeting into complex diatom plastids: functional characterisation of a specific targeting motif.” Plant Mol Biol 64(5): 519-30.
Hallmann, A. (2007). “Algal transgenics and biotechnology.” Transgenic Plant Journal, Global Science Boks.
Kilian, O. and P. G. Kroth (2005). “Identification and characterization of a new conserved motif within the presequence of proteins targeted into complex diatom plastids.” Plant J 41(2): 175-83.
Lusic, I., V. Radonic, et al. (2006). “Is C-reactive protein a better predictor of recurrent carotid disease following carotid endarterectomy than established risk factors for atherosclerosis?” Vasa 35(4): 221-5.
Ma, J. L., E. M. Kim, et al. (2003). “Yeast Mre11 and Rad1 proteins define a Ku-independent mechanism to repair double-strand breaks lacking overlapping end sequences.” Mol Cell Biol 23(23): 8820-8.
Maliga, P. (2004). “Plastid transformation in higher plants.” Annu Rev Plant Biol 55: 289-313.
Marcaida, M. J., J. Prieto, et al. (2008). “Crystal structure of I-Dmol in complex with its target DNA provides new insights into meganuclease engineering.” Proc Natl Acad Sci USA 105(44): 16888-93.
Marenkova, T. V. and E. V. Deineko (2010). “[Transcriptional gene silencing in plants].” Genetika 46(5): 581-92.
Mogedas, B., C. Casal, et al. (2009). “beta-carotene production enhancement by UV-A radiation in Dunaliella bardawil cultivated in laboratory reactors.” J Biosci Bioeng 108(1): 47-51.
Moscou, M. J. and A. J. Bogdanove (2009). “A simple cipher governs DNA recognition by TAL effectors.” Science 326(5959): 1501.
Palmer, J. D., D. E. Soltis, et al. (2004). “The plant tree of life: an overview and some points of view.” Am J Bot 91(10): 1437-45.
Paques, F. and P. Duchateau (2007). “Meganucleases and DNA double-strand break-induced recombination: perspectives for gene therapy.” Curr Gene Ther 7(1): 49-66.
Porteus, M. H. and D. Carroll (2005). “Gene targeting using zinc finger nucleases.” Nat Biotechnol 23(8): 967-73.
Potvin, G. and Z. Zhang (2010). “Strategies for high-level recombinant protein expression in transgenic microalgae: a review.” Biotechnol Adv 28(6): 910-8.
Simon, P., F. Cannata, et al. (2008). “Sequence-specific DNA cleavage mediated by bipyridine polyamide conjugates.” Nucleic Acids Res 36(11): 3531-8.
Steinbrenner, J. and G. Sandmann (2006). “Transformation of the green alga Haematococcus pluvialis with a phytoene desaturase for accelerated astaxanthin biosynthesis.” Appl Environ Microbiol 72(12): 7477-84.
Stoddard, B. L., R. J. Monnat, et al. (2007). “Advances in engineering homing endonucleases for gene targeting: ten years after structures.” Progress in Gene Therapy: AUtologous: 135-167.
Tyler, B. M., S. Tripathy, et al. (2006). “Phytophthora genome sequences uncover evolutionary origins and mechanisms of pathogenesis.” Science 313(5791): 1261-6.
Zaslayskaia, L. A., J. C. Lippmeier, et al. (2001). “Trophic conversion of an obligate photoautotrophic organism through metabolic engineering.” Science 292(5524): 2073-5.

1. A method for targeted modification of the genetic material of an algal cell comprising the steps of:
a) selecting a nucleic acid target sequence in the genome of an algal cell;
b) designing a gene encoding a rare-cutting endonuclease to target this sequence;
c) transfecting algal cells with one or more vectors comprising said gene encoding said rare-cutting endonuclease to obtain its expression within said cell over several generations;
d) selecting the cell progeny of said algal cells having a modified target sequence.

2. A method for targeted modification according to claim 1, wherein said method further comprises:
selecting the transfected algae in which said gene encoding said endonuclease has been stably integrated into the genome

3. A method of claim 1 or 2 wherein said method further comprises:
obtaining mosaic clones comprising cells in which said target sequence contains different types of modifications.

4. A method for targeted modification according to any one of claims 1 to 3, wherein said method comprises transfecting said algal cell with a donor matrix containing a transgene.

5. A method according to claim 4, wherein said modification is a knock-in event of said transgene introduced by homologous recombination with the donor matrix.

6. The method according to any one of claims 1 to 5, wherein said rare-cutting endonuclease is a homing endonuclease.

7. The method of claim 6 wherein said homing endonuclease is an engineered I-Crel.

8. The method according to any one of claims 1 to 5 wherein said rare-cutting endonuclease is an engineered nucleic acid binding domain fused to an endonuclease.

9. The method of claim 8, wherein said engineered binding domain is a TAL effector-like domain or a zinc finger domain.

10. The method of claim 9, wherein said endonuclease is selected from the group consisting of: Fokl, I-Tevl, NucA and ColE7.

11. The method according to any one of claims 1 to 5, wherein said rare-cutting endonuclease is a monomeric TALE-Nuclease.

12. The method according to any one of claims 1 to 11, wherein said one or more vectors used in step c) further comprises a selectable marker and said method further comprises selection of transfected algal cells under pressure of a selective agent.

13. The method according to any one of claims 1 to 11, wherein said one or more vectors used in step c) further comprises a selectable marker included on a different vector and said method further comprises selection of transfected algal cells under pressure of a selective agent

14. The method of claim 12 or 13, wherein said selectable marker is N-acetyltransferase 1 gene (Nat1) conferring the resistance to Nourseothricin.

15. The method of claim 12 or 13, wherein said selectable markers are selected from the group consisting of: Zeocin/Phleomycin and blastidicidin resistance gene.

16. The method according to any one of claims 1 to 15, wherein said gene encoding said rare-cutting endonuclease is placed under control of an inducible promoter.

17. The method according to any one of claims 1 to 16, wherein said algal cell is transformed by a method selected from the group consisting of: electroporation and bombardment methods.

18. The method according to any one of claims 17 wherein algae are selected from the group consisting of Anabaena, Anikstrodesmis, Bottyococcus, Chlamydomonas, Chlorella, Chlorococcum, Dunaliella, Emiliana, Euglena, Hematococcus, Isochrysis, Monochrysis, Monoraphidium, Nannochloris, Nannnochloropsis, Nephrochloris, Nephroselmis, Nodularia, Nostoc, Oochromonas, Oocystis, Oscillartoria, Pavlova, Playtmonas, Pleurochrysis, Porhyra, Pseudoanabaena, Pyramimonas, Stichococcus, Synechococcus, Synechocystis, Tetraselmis, and Trichodesmium.

19. The method of claim according to any one of claims 1 to 16, wherein the algae are diatoms.

20. The method of claim 19, wherein diatoms are selected from the group consisting of: Phaeodactylum, Fragilariopsis, Thalassiosira, Coscinodiscus, Arachnoidiscusm, Aster omphalus, Navicula, Chaetoceros, Chorethron, Cylindrotheca fusiformis, Cyclotella, Lampriscus, Gyrosigma, Achnanthes, Cocconeis, Nitzschia, Amphora, and Odontella.

21. The method according to any one of claims 1 to 20, wherein the mutagenesis is increased by transfecting the cell with a transgene coding for a catalytic domain having exonuclease activity.

22. The method of claim 21, wherein said catalytic domain has 3′-5′ exonuclease activity.

23. The method of claim 21, wherein said catalytic domain has TREX exonuclease activity.

24. The method of claim 21, wherein said catalytic domain has TREX2 activity.

25. The method of claim 24, wherein said catalytic domain is encoded by a single chain TREX2 polypeptide.

26. The method according to any one of claims 21 to 25, wherein said additional catalytic domain is fused to said rare-cutting endonuclease, optionally by a peptide linker.

27. The method according to claims 1 to 26, which comprises a further step of inactivating the gene encoding the rare-cutting endonuclease in the modified progeny cells.

28. The method according to claims 1 to 27, which comprises selecting the algal cells that display modifications in the target gene, in multi-copy genes or more than one allele.

29. A genetically modified algal cell obtained by the method of any one of claims 1 to 28.

30. A genetically modified algal cell of claim 29 in which a UDP-glucose pyrophosphorylase gene is inactivated.

31. The genetically modified algal cell of claim 30 wherein said UDP-glucose pyrophosphorylase gene has at least 80% identity sequence with SEQ ID NO: 41.

32. The genetically modified algal cell of claim 30 or 31 obtained using a TALE-nuclease.

33. The genetically modified algal cell of claim 32, wherein the TALE-nuclease targets a sequence of SEQ ID NO: 44.

34. The genetically modified algal cell of claim 33, which is a Phaeodactylum tricornutum strain as deposited within the Culture Collection of Algae and Protozoa (CCAP, Scottish Marine Institute, Oban, Argyll PA34 1QA, Scotland) on May 29^{th}, 2013 under CCAP 1055/12 and depositor's strain number pt-37-7A1.

35. The genetically modified algal cell of claim 29 in which a putative elongase gene is inactivated.

36. The genetically modified algal cell of claim 35, wherein said putative elongase gene has at least 80% identity sequence with SEQ ID NO: 52.

37. The genetically modified algal cell of claim 35 or 36 obtained using a TALE-nuclease.

38. The genetically modified algal cell of claim 37, wherein the TALE-nuclease targets a sequence of SEQ ID NO: 55.

39. A genetically modified algal cell of claim 38 which is a phaeodactylum tricornutum as deposited within the Culture Collection of Algae and Protozoa (CCAP, Scottish Marine Institute, Oban, Argyll PA34 1QA, Scotland) on May 29, 2013 under CCAP 1055/13 and depositor's strain number pt-42-11B5.

40. A genetically modified algal cell, characterized in that its genome comprises targeted modification in several alleles or homologous genes.

41. A genetically modified algal cell, characterized in that its genome comprises a transgene encoding a TALE-Nuclease.

42. A genetically modified algal cell, characterized in that its genome comprises transgenes encoding a TALE-Nuclease and a TREX exonuclease.

43. A genetically modified algal cell, characterized in that its genome comprises transgenes encoding a meganuclease and a TREX exonuclease.

44. A genetically modified algal cell, characterized in that its genome comprises a TALE-Nuclease-induced targeted modification.

45. The genetically modified algal cell according to any one of claims 29 to 34, wherein its genome includes a gene encoding a rare-cutting endonuclease which expression is under control of inducible promoter.


SEQUENCE LISTING (140 sequences)

SEQ ID NO: 1 - DNA, 3655 bp, artificial sequence
  synthetic polynucleotide pCLS17038 (PTRI20)
gttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtacgtgtacatcatacgtaaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaattaattaatgcggcgcgcctacacagcggccttgccaccatggccaataccaaatataacgaagagttcctgctgtacctggccggctttgtggacggtgacggtagcatcatcgctcagattaaaccaaaccagtcttgtaagtttaaacatgctctaagcttgacctttcaggtgactcaaaagacccagcgccgttggctgctggacaaactagtggatgaaattggcgttggttacgtacgtgattctggtagcgtttcccattactacttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctgggtagcctgagcgagaagaagaaaacctccccggcggccggtgattcctctgtttctaattccgagcacattgctcctctgtctctgccttcctctcctccatctgttggttctaacaaaaaattcctgctgtatcttgctggatttgtggattctgatggctccatcattgctcagataaaaccaaatcaatctaccaagttcaaacaccagctctggttgacctttcaagtcactcagaagacacaaagaaggtggttcttggacaaattggttgatcgtattggtgtgggctatgtcagagacagaggctctgtgtcagactaccatctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagtaactcgagcgctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttgtgagtcctgcaggtacgtttaaacacgattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 2 - DNA, 24 bp, artificial sequence
  synthetic polynucleotide PTRI20 target
gttttacgttgtacgacgtctagc

SEQ ID NO: 3 - DNA, 4246 bp, artificial sequence
  synthetic polynucleotide pCLS16604
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtaccccgctttggtttcacagtcaggaataacactagctcgtcttcaccatggatgccaatctcgcccattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaagtcaagtcgaggtcgatccatatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagcggccgacggtatcgataagcttgatatcgaattcctgcagcccgggggatccactagttctagagcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 4 - DNA, 5428 bp, artificial sequence
  synthetic polynucleotide pCLS003
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 62 bp, artificial sequence
  synthetic polynucleotide Deep seq PTRI20_for
ccatctcatccctgcgtgtctccgactcagnnnnnnnnnncggttgtcatggatagcggagc

SEQ ID NO: 6 - DNA, 53 bp, artificial sequence
  synthetic polynucleotide Deep seq PTRI20_Rev
cctatcccctgtgtgccttggcagtctcagccccagacgattcgaagtcgtcc

SEQ ID NO: 7 - DNA, 4599 bp, artificial sequence
  synthetic polynucleotide pCLS18296
ctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccgctacgacgctcgctccagctggggcttccttactatacacagcgcgcatatttcacggttgccagaattaattaagtaggcgcgccactagcgctgtcacgcgtctccatgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcgactcctccacagaccggtctggatgttccttactccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcggccgactgataactcgagcg

SEQ ID NO: 8 - DNA, 30 bp, artificial sequence
  synthetic polynucleotide Screen colony meganuclease For
ttaacaattgaatctcgcctattcatggtg

SEQ ID NO: 9 - DNA, 35 bp, artificial sequence
  synthetic polynucleotide Screen colony meganuclease Rev
tagcgctcgagttactaaggagaggactttttctt

SEQ ID NO: 10 - DNA, 20 bp, artificial sequence
  synthetic polynucleotide Screen colony SCTREX2 For
aatctcgcctattcatggtg

SEQ ID NO: 11 - DNA, 21 bp, artificial sequence
  synthetic polynucleotide Screen colony SCTREX2 Rev
ccagaccggtctgtggaggag

SEQ ID NO: 12 - DNA, 3654 bp, artificial sequence
  synthetic polynucleotide pCLS17181 (PTRI02)
gttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtacgtgtacatcatacgtaaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaattaattaatgcggcgcgcgcctacacagcggccttgccaccatggccaataccaaatataacgaagagttcctgctgtacctggccggctttgtggacggtgacggtagcatcatcgctcagattaaaccaaaccagtcttgtaagtttaaacatcagctagctttgacctttcaggtgactcaaaagacccagcgccgttggtttctggacaaactagtggatgaaattggcgttggttacgtacgtgatcgcggtagcgtttccgactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtgattcctctgtttctaattccgagcacattgctcctctgtctctgccttcctctcctccatctgttggttctaacaaaaaattcctgctgtatcttgctggatttgtggattctgatggctccatcattgctcagataaaaccacgtcaaacctacaagttcaaacaccagctctccttgacctttaaagtcactcagaagacacaaagaaggtggttcttggacaaattggttgatcgtattggtgtgggctatgtctacgactctggctctgtgtcatactaccagctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagtaactcgactagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttgtgagtcctgcaggtacgtttaaacacgattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 13 - DNA, 24 bp, artificial sequence
  synthetic polynucleotide PTRI02 target
ttttgacgtcgtacggtgtctccg

SEQ ID NO: 14 - DNA, 62 bp, artificial sequence
  synthetic polynucleotide Deep seq PTRI02_for
ccatctcatccctgcgtgtctccgactcagnnnnnnnnnntcagctccattggaatgttggc

SEQ ID NO: 15 - DNA, 53 bp, artificial sequence
  synthetic polynucleotide Deep seq PTRI02_Rev
cctatcccctgtgtgccttggcagtctcagccctccgaccagggaacttactc

SEQ ID NO: 16 - DNA, 6329 bp, artificial sequence
  synthetic polynucleotide pCLS17205
ctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccgctacgacgctcgctccagctggggcttccttactatacacagcgcgcatatttcacggttgccagaattaattaagtaggcgcgccactagcgctgtcacgcgccaagccgccaccatggtttaacatggccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagct

SEQ ID NO: 17 - DNA, 6335 bp, artificial sequence
  synthetic polynucleotide pCLS17208
ctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccgctacgacgctcgctccagctggggcttccttactatacacagcgcgcatatttcacggttgccagaattaattaagtaggcgcgccactagcgctgtcacgcgccaagccgccaccatggtttaacatggccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataatgataactcgagcgatcctctagacgagctcctcgagcctgcagcagctgaagct

SEQ ID NO: 18 - DNA, 45 bp, artificial sequence
  synthetic polynucleotide YFP target
tgaaccgcatcgagctaagggcatcgacttcaaggaggacggcaa

SEQ ID NO: 19 - DNA, 61 bp, artificial sequence
  synthetic polynucleotide Deep seqYFP_for
ccatctcatccctgcgtgtctccgactcagnnnnnnnnnnctgcaccaccggcaagctgcc

SEQ ID NO: 20 - DNA, 47 bp, artificial sequence
  synthetic polynucleotide Deep seq YFP_Rev
cctatcccctgtgtgccttggcagtctcagcctcgatgttgtggcgg

SEQ ID NO: 21 - DNA, 6629 bp, artificial sequence
  synthetic polynucleotide pCLS20885 (TALEN TP07)
ggtaccataactagttcggcgcgccgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctccccctgcagggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaactt

SEQ ID NO: 22 - DNA, 6647 bp, artificial sequence
  synthetic polynucleotide pCLS20886 (TALEN TP07)
cgcgccgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctccccctgcagggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcgg

SEQ ID NO: 23 - DNA, 49 bp, artificial sequence
  synthetic polynucleotide TP07 target
tgactttcctcccatgttaggtccagtgacaagaaggaatgaggatgca

SEQ ID NO: 24 - DNA, 4436 bp, artificial sequence
  synthetic polynucleotide pCLS17714 (NAT LHCF9)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaattaattaagtaggcgcgccatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctcccgtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 59 bp, artificial sequence
  synthetic polynucleotide Deep seq TP07_for
ccatctcatccctgcgtgtctccgactcagnnnnnnnnnnggaagtgagttgcaaacac

SEQ ID NO: 26 - DNA, 50 bp, artificial sequence
  synthetic polynucleotide Deep seq TP07_Rev
cctatcccctgtgtgccttggcagtctcagcttcaagatgatatgaactt

SEQ ID NO: 27 - DNA, 6629 bp, artificial sequence
  synthetic polynucleotide pCLS20726(TALEN TP15)
ggtaccataactagttcggcgcgccgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctccccctgcagggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaactt

SEQ ID NO: 28 - DNA, 6647 bp, artificial sequence
  synthetic polynucleotide pCLS20727 (TALEN TP15)
cgcgccgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctccccctgcagggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcgg

SEQ ID NO: 29 - DNA, 49 bp, artificial sequence
  synthetic polynucleotide TP15 target
ttgggtcttgaagggatgttgtcgggaaccacgttggccatggagtgga

SEQ ID NO: 30 - DNA, 61 bp, artificial sequence
  synthetic polynucleotide Deep seq TP15_For
ccatctcatccctgcgtgtctccgactcagnnnnnnnnnnaatgcccaaagtatacactgt

SEQ ID NO: 31 - DNA, 51 bp, artificial sequence
  synthetic polynucleotide Deep seq TP15_Rev
cctatcccctgtgtgccttggcagtctcagaattcattatctccgactctc

SEQ ID NO: 32 - DNA, 4470 bp, artificial sequence
  synthetic polynucleotide pCLS19635 (MatriX PTRI02)
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagattcagttgcacccacgaatgagggccagaacacccacaaaattgtcgtacctatcaaggccatcagatccgaaaccttatccggttcgggtgtacctagttcttctttgattctgtctttaccgtcagatcgtcctctgcccatggccagactagcggctagaccgaagtacgcgcctgtgcagtttcgcataaggtcaacagcatgagaagtgcgacgatgctgctccagacgtacagcccttaagcgatcgatcacgcagacaaggcatataatggtgacttaccaaagcagtgaatcgtcatggatccacccacgtcttcggctccgaccattccaaatacgaaaaacaccttattgagtgcataaaagaaggattgcgatagggcgatgagcatcatctgcaggggcgtggccgttccaataagcgccccgaaagagatcataagagtagccgcgctgaactcggcatcaatcagtgtctccatggacaatggcataggccacgatgtatcctcaccgtcatcgcctttaagagtgcgcattatcagctccattggaatgttggcctccatcgcaaggatactcaacatcatggtgaatccaaccgcgccaatgccgtagtttttcagaaacgtcatcaggtagccaaagccgagcaacaacatggccatgatatctcgaaaggcgatgtatgttttgacgtcgtttaaggcgcgccggaccgcggccgcaattacggtgtctccgtcgaatacgttgttccaaataaatagaacaatagaaaggcggtctggagcgccacgaaaaggttcgcgatggtgacgacacctttggcggacggagcatgcgagccattatccgagctcaacagtagcgaagtcgtttccataggagcactcatagtctgctctcggatgaatggagtaaataaaagatcggaaagagaagagtaagttccctggtcggagggaaggacattgaccaacggcgtatcttttaagattgtttgtgtacacgtccgggaaggtgtatcttttcgtgagaatgatgccctctttcctccgtgttggatttcggcagtgggcacccgaaattgtttaaaaattccgagacgtaaaatttcgggtttacttcgaatgttaatttatcgtcgagagatcttgagtagcggtgcgactttgctttttacaaggtttcattaaagcgaatcaattgcaccattcggtgttctcgttttggtagagaagaatcaaacgagaaagacatcgagttttttaccgttcgctgctgtacattggttgcggttctattcacggtcatctagatagtacagcctatgatttgattcacattcttttcgacaatcgaacaactggcgactagaggagactgaaaatcgggatcgaggtgatcaaaagaaaggtgtatggagtctttctatgtcaaattgtgttaaaatctattgttcgtatgtacaagaactagcaagagcagatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 33 - DNA, 27 bp, artificial sequence
  synthetic polynucleotide PTRI02_HGT_Left_For
ccggccagagtcgaattggccacgtgg

SEQ ID NO: 34 - DNA, 22 bp, artificial sequence
  synthetic polynucleotide Insert_HGT_Left_Rev
aattgcggccgcggtccggcgc

SEQ ID NO: 35 - DNA, 22 bp, artificial sequence
  synthetic polynucleotide PTRI02_HGT_Right_For
ttaaggcgcgccggaccgcggc

SEQ ID NO: 36 - DNA, 27 bp, artificial sequence
  synthetic polynucleotide PTRI02_HGT_Right_Rev
gacgacgacgaaaacgtcttgcgtccg

SEQ ID NO: 37 - DNA, 4419 bp, artificial sequence
  synthetic polynucleotide pCLS19773 (MatriX PTRI20)
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatgctgtttggaacatctactccacgctgaaccggaacccaccgctgatttctttacggcgctacagtctttacctgattggctgtcagaacgggctgtggtcatgcctgtggtgcagattttattaaacgaaaagatttcgcaacggtttcccaccgcagtccttatgatggatttttacgtatgtacgtgcacaactgtaatgctcactcggtaccagaatatcgggtagcaatgacgcttatgttgagattccttacacatctcaattgttcgttcttttgttccgtttgacgattgcagtgactatggttatcatttcctattcacataacgtggtgaattccattcaacgtcggtttgatgatgacgacacaaacgatacaattgcaaccaaatcgttgattcctctctacttgggcgtcgcatactttgcactgcgtgaagtcgtccaaatcatgtcgctgttgagcctcaaggtcttcaagctatggctttacgatccaagtaattatctcaacgtggcgttcatcgctttggttttgagttggacggttgtcatggatagcggagctggcgatcgcgacacttttcgagtcggcgctgccgtgtcggtcaccattttatgggtcaagctcctcgcatatctacgcaatatgctgattgacttcgccgtctttgttggtggggtgttttacgttgtttaaggcgcgccggaccgcggccgcaattacgacgtctagcagcctttctcttggctttaggacttattctggttgcgttcgcgcaaatgttctacacggtttttcaacagacagactactgtcgaaatcaaccgcaaaatgatttggactacgacctgatactggcagaaactcggtgcgatgccagcacgctacgtccgtactgtggcttttggacttcttttctgagcgtatacaccatgttgctaggagaagtagacgaggacgacttcgaatcgtctggggtggccatggcactattcgtcatctttatgttcttggtcgtaattttgcttgcaaacgtgttaattgctatcgttacggacagttacaaagtgattcaatatcagcgtgctgccatagtgttttggacaaatcgcttggatttcgtcgcagagatggatgccatagcaaatggaccctggaagagccgcgtcaagaaatccttgggcatgttggatcaagatgaagatggagcgcaacaacgggatgtctttggaaaggatctatggaaacagattatggatttgtttgaagacgactcgtttgacgggatgtcaacggtggactttattgctttcgcgctccttcgggtggttgcttgcgtctttatcattcctatgtggctgctactgggcattgttactgttgggtggttttggcctccacaagttcgggaagcggtcttcacaagtaaggtgtcaatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 38 - DNA, 28 bp, artificial sequence
  synthetic polynucleotide PTRI20_HGT_Left_For
gcagcgtacgcagccatagtccggaacg

SEQ ID NO: 39 - DNA, 26 bp, artificial sequence
  synthetic polynucleotide PTRI20_HGT_Right_For
tgttttacgttgtttaaggcgcgccg

SEQ ID NO: 40 - DNA, 28 bp, artificial sequence
  synthetic polynucleotide PTRI20_HGT_Right_Rev
ccgcatctcaatcacgtcttgttgaagc

SEQ ID NO: 41 - DNA, 3613 bp, Phaeodactylum tricornutum
  MISC_FEATURE (1)..(3613): UDP-Glucose-Pyrophosphorylase/ Phosphoglucomutase (UGP/PGM) (PHATRDRAFT_5044)
ttcgaaagacgaacaacgagcctcccgaatatcctacggttggttgcttaataacattgctttgacaactcaagatgccttctttcgatcccattcgtgcaaaaatggaagccggaggctgtgctccatcggcgattgccgccttcgagtcgacctatggtagtctcgtctcgggtgattccggaatgattttggaagactctattgcgcccgtcccccagctggacaagaccgcggagctggatattgcacccaacgccacccttcttgccgagacggtagttctcaaactcaatggtggactcggcacgggtatgggtctggacaaggccaagtccctgttgccagtcaagggggacgacacctttttggatttgaccgccaaacaagtcattcaaatgcgtaaggaatacggtttgaacgtcaagtttatgctcatgaattcgttttctacttccgacgataccttgagctttttgagttccaaataccctgatcttgcttccgaggaaggtttagaaatgatgcaaaataaggtccccaagttgaacgcggagactctcgagccggcatcttgtgaatccgatccggaaaatgagtggtgtccgccgggacacggtgacttgtacgcggccttggttggctctggtcgtcttgatgccctgctcaaggaagggttcaaatatatgtttgtctccaattcggacaaccttggtgctagcctggaccttgaaattctgacttactttgccgagaagaatgtacccttcttgatggagtgctgcgaacgtacagaaaacgacaaaaagggagggcacttggccgtccgcaaatccgatggacaacttattcttcgggaatctgctatgtgcgctgaagaggatgaagatgcattcagtgatatcagcaagcaccgctttttcaacaccaacaatttgtgggttcgtctcgataaactcaaggagatcatcgaccgcaatggcggctttattcctctgcccatgatcaaaaacaaaaagacggtcgaccccaaggacgactcgtcgaccccggtactgcagttggaaaccgctatgggtgccgctattgaatgtttcgaaggcgccagcgcggtggttgttcctcgcacacgctttgcgcccgtcaaaaagtgcagcgatctgctcttgctgcgctccgatgcatacttgctcgtggaccacaagccggtactcaatccagcctgcaacgggagcgcgcccgtgatcaatctcgacagcaaactatacaagctggtcggcgccttggaagaagcaacccaggacggcattccgtccctcgtcaagtgcgacaaattgactatcaagggtttggtccggatgtcgaaaaagaccaagtttgtgggtgatgtcaagattgtcaactcgagcgccgaatctaagtttgtgcccaccggtgaagtaacaggggaacacgatctgacgtctaatgctggtcttggcaagctaaagcccacctctgtttcaacagcaccaattgcgggacaaaagcctggtacttcaggactccggaagaaggttgccgaattcaagaaggaaaactaccttaacaattttgtacaagctgcttttgacgccatcaaggccagtggtacggacatatcgaaggggtccttggtaattggtggtgatggtcgctacttcaaccctgaagcaatccaaatacttattcagatgggtgttgctaacggcgtcagacgtttctggattggacaggacggcctcttgtcgacacccgccgtttctgcgatcattcgggaaggcggcccgcgttggcaaaaggcatttggagcctttattttgacggctagtcacaatcccggtggcccaacggaagattttggtatcaagtacaactgcgaacatggtgagcccgctccggagaggatgacggatgaaatttacgccaacacaacgacgattaagtcctacaagatttgtaaggaattccccaacattgacattggcgctgcgggccactccaagatcatgtctgacgacggcagcgccgaagtcaatattgaagtaattgattccaccgaagctcacgtcaagttgttgaaatctatttttgatttctcggccatcagagggctgttggatcgccccgacttttccatggtctacgacgccatgcacggtgtcaacgggccgtacgtaaaaaaagtattctgcgatattctggggcaggacctctccgtcacactgaactgtgtccccaaggacgacttcaacggaggccatgccgaccccaacctcacgtacgccaaagagcttgttgccgtcatggggcttaatcgcaagggcgaaaagatcgatatgggcggacgtcctattcccagctttggtgcggccgccgacggcgacggagaccgcaacatgattctgggcacacagttttttgtcagtccgtccgattccttggcagtcattgttgccaacgccgacaccattccattcttccgcacgcaaggtggactcaagggcgtcgcgcggtccatgccaacgtccggcgccgtcgatctcgtcgccaaggacctgaactacagtttgtttgaaacacctacgggatggaaatacttcgggaacctgatggattccaaagagctttttgacggtgccgaatacactccgtttatttgtggggaagaatcgttcggcacaggctccgatcacattcgcgaaaaggacggactttgggccgtgctggcttggctcagcattttggcgcacgccaatactaacagcctaagtgacacactggtgaccgtggaagacattgtcaaggctcattgggcaaagtacggacgcaactactacagccgctgggatttcgagaacatgaatgcgaccaaggcgaacgccatgatggacaagatgcgggcggaaacagacgcgaacacgggcaagacggtgggcaagtactcgatcgaaaagtccgacgactttgtgtacgtggatcccgtggacggctcggtggccaagaagcaggggatgcggttcctaatgacggatggctcgcggattattttccgtttgagtggcacggcgggcagtggcgccacggtccgcatgtacatcgaacagtacgaaccgacgaagattgacatggtggcttcagaggctttggcagatttgattcgagtcgcactggatttatctgacctcaagggattcctcggaactgaagaaccaaccgtaattacgtaactgatgttcgagctctggcaacacgtcctgctaggtctcagtgtggctaactaaacgagccagccagaacagtttcctccgtctgatatatgaatgatgtgactcgctcaggaatcgattcgtaattgtcgagtagagcaacttaatagtgcaacaacgatagccctagtgcaaaatcctcgtctcgtttcgatgggttcatgcatcctaatgcaagctgaatatttcgttgtctatccgagtaatacaaagagaaaattcggtatttgggatgagcaggggtgaaattttcgctatttgggaaaaatcacactgtttctaagtgtttttattttcgcgggaaatactttctaagtaatcttttt

SEQ ID NO: 42 - DNA, 5922 bp, artificial sequence
  synthetic polynucleotide pCLS19745 (TALEN UGP)
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 43 - DNA, 5940 bp, artificial sequence
  synthetic polynucleotide pCLS19749 (TALEN UGP)
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 44 - DNA, 50 bp, artificial sequence
  synthetic polynucleotide UGPase target
tgccgccttcgagtcgacctatggtagtctcgtctcgggtgattccggaa

SEQ ID NO: 45 - DNA, 62 bp, artificial sequence
  synthetic polynucleotide Deep seq UGP_For
ccatctcatccctgcgtgtctccgactcagnnnnnnnnngttgaatcggaatcgctaactcg

SEQ ID NO: 46 - DNA, 52 bp, artificial sequence
  synthetic polynucleotide Deep seq UGP_Rev
cctatcccctgtgtgccttggcagtctcaggacttgtttggcggtcaaatcc

SEQ ID NO: 47 - DNA, 23 bp, artificial sequence
  synthetic polynucleotide Primer UGP For
gttgaatcggaatcgctaactcg

SEQ ID NO: 48 - DNA, 22 bp, artificial sequence
  synthetic polynucleotide Primer UGP Rev
gacttgtttggcggtcaaatcc

SEQ ID NO: 49 - DNA, 20 bp, artificial sequence
  synthetic polynucleotide Screen TALEN_For
aatctcgcctattcatggtg

SEQ ID NO: 50 - DNA, 21 bp, artificial sequence
  synthetic polynucleotide ScreenHA_Rev
taatctggaacatcgtatggg

SEQ ID NO: 51 - DNA, 21 bp, artificial sequence
  synthetic polynucleotide Screen Stag_Rev
tgtctctcgaacttggcagcg

SEQ ID NO: 52 - DNA, 747 bp, Phaeodactylum tricornutum
  MISC_FEATURE (1)..(747): elongase putative protein (PHATRDRAFT_49867)
atggaagcgcatccgttggttcccattggcgcctgcctactctacggactcttgatggtggcgggacaggcctactttcgcacacgcgaaccactccgggcgcggacctccctcgcggcctggaatctctttctggccctcttttccctcgtcggcatgctccggacctttccccagcttgtacacaacctcgcgacgctcacgctccgggaaaatctctgcgccaatccgcaagccacctacggatccggatccaccggattgtgggtacaactctttattctgtccaaattccctgaactcattgatacagtattcatcattgtcaacaagaagaaactcatcttcttacactggtaccatcacattacggtcctcctctactgctggcacagttacgtcaccaaatccccgccgggaattttctttgtcgtcatgaactacaccgtccacgcctccatgtacggatactactttctcatggccatccgagcccgaccccgttggctcaatcccatgattgtcacgactatgcaaatatcgcaaatggtcgtgggcgtcgccgtcaccctccttggcttttactactcggcacgtgccgccgaccaccaatcctgtcgaattaaacgggaaaacaacaccgccgcctttgtcatgtacggatcctatctatttctctttctgcagttctttgtgggacgctacgttggcacccaatccccagtcgcgtccaaaaagacggcctaa

SEQ ID NO: 53 - DNA, 5922 bp, artificial sequence
  synthetic polynucleotide pCLS19746 (TALEN Elongase)
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 54 - DNA, 5940 bp, artificial sequence
  synthetic polynucleotide pCLS19750 (TALEN Elongase)
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 55 - DNA, 49 bp, artificial sequence
  synthetic polynucleotide Elongase target
tcttttccctcgtcggcatgctccggacctttccccagcttgtacacaa

SEQ ID NO: 56 - DNA, 58 bp, artificial sequence
  synthetic polynucleotide Deep seq Elongase_For
ccatctcatccctgcgtgtctccgactcagnnnnnnnnnaagcgcatccgttggttcc

SEQ ID NO: 57 - DNA, 52 bp, artificial sequence
  synthetic polynucleotide Deep seq Elongase_Rev
cctatcccctgtgtgccttggcagtctcagtcaatgagttcactggaaaggg

SEQ ID NO: 58 - DNA, 19 bp, artificial sequence
  synthetic polynucleotide Primer elongase For
aagcgcatccgttggttcc

SEQ ID NO: 59 - DNA, 22 bp, artificial sequence
  synthetic polynucleotide Primer elongase Rev
tcaatgagttcactggaaaggg

SEQ ID NO: 60 - protein, 50 aa, artificial sequence
  plastid targeting sequence
MARTRHGARLARCAFVWLWVASSTTAFTTTSPRLAAHFRSASRTQRTTTT

SEQ ID NO: 61 - protein, 50 aa, artificial sequence
  plastid targeting sequence
MRSFCIAALLAVASAFTTQPTSFTVKTANVGERASGVFPEQSSAHRTRKA

SEQ ID NO: 62 - protein, 50 aa, artificial sequence
  plastid targeting sequence
MKLSTAALFFIPAVVAFAPPQAAFRSNPALFATETAAEKTTFSKMPASVK

SEQ ID NO: 63 - protein, 50 aa, artificial sequence
  plastid targeting sequence
MAKFSLAILAALVATASAFVASPTTSSASTALRATQEGVWDPLGLMTLGT

SEQ ID NO: 64 - protein, 50 aa, artificial sequence
  plastid targeting sequence
MKSSAVLALAMAGSTAAFAPTSSTQASTSSTSLQAAMPDRLWNTMVDKTE

SEQ ID NO: 65 - protein, 50 aa, artificial sequence
  plastid targeting sequence
MKITALCLTALVSASHAFAPSTPSSASSSARALSTESTSDVPLLQIKEKV

SEQ ID NO: 66 - protein, 50 aa, artificial sequence
  plastid targeting sequence
MKYRTSILFSALATSATAFAPTQSITRTLTQTNMADFNKDDFLSFKEKDK

SEQ ID NO: 67 - protein, 50 aa, artificial sequence
  plastid targeting sequence
MKLFTIFLPLVLVGTAAGFASGPFSKKASPSPEVSIESMPGIVAPTGFFD

SEQ ID NO: 68 - protein, 50 aa, artificial sequence
  plastid targeting sequence
MMKLALIASLVAGAAAFAPASKQASSSALKAFENEAGVIQPTGFFDPFGL

SEQ ID NO: 69 - protein, 50 aa, artificial sequence
  plastid targeting sequence
MKFSLTLVAVFVSGSAAFVPNSFSATARTDALRMTSPGYEIESESSGARR

SEQ ID NO: 70 - protein, 50 aa, artificial sequence
  plastid targeting sequence
MKLALVFSLFATAAAFSQEASRREALTKGAAAFGAAFLPVAANAAVGESP

SEQ ID NO: 71 - protein, 50 aa, artificial sequence
  plastid targeting sequence
MKIIPTVTSLALLAISIRAFTPTLAPRHTHASSKTWAASLEEPAYEGTID

SEQ ID NO: 72 - protein, 50 aa, artificial sequence
  plastid targeting sequence
MKSFQLLTLFALIAASLAFAPNQAPQQVAKAAFTKAAGALPVAALAAPAF

SEQ ID NO: 73 - protein, 50 aa, artificial sequence
  plastid targeting sequence
MTVSTYFIFFFTLGRGTAAFSPSLGASLTRSTAVPRFALSPSSNDDGIHS

SEQ ID NO: 74 - protein, 50 aa, artificial sequence
  plastid targeting sequence
MMHRKIALVAGLWFCLLGSSCHGFGLLGGAAHSRTVRWPQQWAVSSPIGP

SEQ ID NO: 75 - protein, 50 aa, artificial sequence
  plastid targeting sequence
MHHFAKVILLVCVAMLAAVYTEAFATPSRSVQSSAVSITNSMPFRTSALN

SEQ ID NO: 76 - protein, 50 aa, artificial sequence
  plastid targeting sequence
MMQQQQQQQHAAQRRAGVGTILLSLWFVPAATAFAPSTPLAFRTQTPVVT

SEQ ID NO: 77 - protein, 50 aa, artificial sequence
  plastid targeting sequence
MYRAQQYCRSRTLFIHYAIVLVLTRYCSAFCSLEPVLRPSRWISNRSKSL

SEQ ID NO: 78 - protein, 50 aa, artificial sequence
  plastid targeting sequence
MKLNIAAIALFAASASAFAPRFASPRSHATVLSAVLEERTGQSQLDPAVI

SEQ ID NO: 79 - protein, 50 aa, artificial sequence
  plastid targeting sequence
MLRSLAFTASVALLFSLDPLLAFAPIRTTTSVASPSGVSIRAQTGDQLFA

SEQ ID NO: 80 - protein, 50 aa, artificial sequence
  plastid targeting sequence
MKLPWLGPSAAALLLSSQTMAFLPSSLPSQSARNAGVTLQEKPSASDSSF

SEQ ID NO: 81 - protein, 50 aa, artificial sequence
  plastid targeting sequence
MAPLRTTFALLLSLVSASAFAPVQNVARKQTSVSAFKIDPQLYDDAVSDW

SEQ ID NO: 82 - protein, 50 aa, artificial sequence
  plastid targeting sequence
MLFKPSTLLALFAVAGTTLAFAPRSTTTPLTSTTRGSASSSVTTLAMSGI

SEQ ID NO: 83 - protein, 50 aa, artificial sequence
  plastid targeting sequence
MKIAVVAFFVIAQCGAFAPAHYSRTVTSSTLLGAKEKGGTSKELDLPCAD

SEQ ID NO: 84 - protein, 50 aa, artificial sequence
  plastid targeting sequence
MEKWGFSRGQVPLLLSVIALSFVLLPTTNSFQTSTGQSQPQRLPASSASL

SEQ ID NO: 85 - protein, 50 aa, artificial sequence
  plastid targeting sequence
MKIALLPVVFSAISVRAFLPTRPSPATQYFRYGPRLATASLSQAAGAAVS

SEQ ID NO: 86 - protein, 50 aa, artificial sequence
  plastid targeting sequence
MKTSAIVAILAVSGASAFTPNTNAPQQLTKVGATAELDNMLGVDIETGKK

SEQ ID NO: 87 - protein, 50 aa, artificial sequence
  plastid targeting sequence
MKCIAAIALLATTASAFNAFGAAKKAAPKKPVFSIETIPGALAPVGIFDP

SEQ ID NO: 88 - protein, 50 aa, artificial sequence
  plastid targeting sequence
MKKILKRSTICCCLGLYTGSPCSYAFRPPPISEEIGCSPTLAIFLKEDCH

SEQ ID NO: 89 - protein, 50 aa, artificial sequence
  plastid targeting sequence
MNTQFVSALLLASAAITNGFAFVNTHRYTASTTALEAGVKIYYSSSTGNT

SEQ ID NO: 90 - protein, 50 aa, artificial sequence
  plastid targeting sequence
MKVFLSFVALLVLSLTQAFMPVSKPSFGRVGGTVYMAKEMTPEEEEIAVE

SEQ ID NO: 91 - protein, 50 aa, artificial sequence
  plastid targeting sequence
MRISSKLMAVWLTTTVGIASAFQPATPSRIRQSPRTTVVLAMSDDWKPPA

SEQ ID NO: 92 - protein, 50 aa, artificial sequence
  plastid targeting sequence
MTTNRVLFLAILALTCLRTRAFLPVTTFVSRTVAGSHGVHSLSMADMADD

SEQ ID NO: 93 - protein, 50 aa, artificial sequence
  plastid targeting sequence
MAILSRTLVGVLTATFACIPHETNAFLKPHLGTVSSLVSIGEPSSTRLHI

SEQ ID NO: 94 - protein, 50 aa, artificial sequence
  plastid targeting sequence
MKSFIACAFILASAAAFAPQPGSPVLRSTSALSASPSDDRSPNPIIKVMA

SEQ ID NO: 95 - protein, 50 aa, artificial sequence
  plastid targeting sequence
MRNITKLRLLAVLAVAASPIRTSQAFAPSVSRKQTNCHKTPLTTSVSIKN

SEQ ID NO: 96 - protein, 50 aa, artificial sequence
  plastid targeting sequence
MRSQILLCFRILFLFLLPTTTIAFSTHPKFRARRKQPCASSQLAVTDKHE

SEQ ID NO: 97 - protein, 50 aa, artificial sequence
  plastid targeting sequence
MVQTLPVFRSLSVMAIGSFLLALGPSPSADAFAFSTRLPTQSLSLRSTST

SEQ ID NO: 98 - protein, 50 aa, artificial sequence
  plastid targeting sequence
MIPTTSVASHRSVVLVGLACTTLLLAVLSPSSTEAFAPSAHRYSANQAVP

SEQ ID NO: 99 - protein, 50 aa, artificial sequence
  plastid targeting sequence
MPGRVVPLSLLAVVCASDLFRSTLAFHPKVSTSSRTIPPSTTQLHAFGFL

SEQ ID NO: 100 - protein, 50 aa, artificial sequence
  plastid targeting sequence
MKLAVIATLLATASAFSIQAEFSKVAKGAAAVGVGAVIAAAPALAGDVGA

SEQ ID NO: 101 - protein, 50 aa, artificial sequence
  plastid targeting sequence
MKFSSAAATTVGLLLSGHAPMIFSFVTPPSRFASGHQASGSERSIISHST

SEQ ID NO: 102 - protein, 50 aa, artificial sequence
  plastid targeting sequence
MKFAVFASLTATAAAFAPTAFVPSNLRGVAPSASSLNMALKEGQTPIIIG

SEQ ID NO: 103 - protein, 50 aa, artificial sequence
  plastid targeting sequence
MKFLSASIALLACATSVEAFNANKAFRFGAKAMPEVSSESATSALSAGGA

SEQ ID NO: 104 - protein, 50 aa, artificial sequence
  plastid targeting sequence
MKFTIVSLAAVVASASAFAPATKSVRSVSALNVWGDKDYLIAPSILSADF

SEQ ID NO: 105 - protein, 50 aa, artificial sequence
  plastid targeting sequence
MRLTAGASILLASSAHAFTNPAFFPRTATFASTSSYTSALILKMGVDQDE

SEQ ID NO: 106 - protein, 50 aa, artificial sequence
  plastid targeting sequence
MRLTLTIQVALTLLLLPSSTVWAFRTSPATQVFSRSRPMRSVASSSSSFS

SEQ ID NO: 107 - protein, 50 aa, artificial sequence
  plastid targeting sequence
MRLQSGCVLTLLAATFYPSTQAFSIFSVGPSFASSSFASRERSDVSRKSY

SEQ ID NO: 108 - protein, 50 aa, artificial sequence
  plastid targeting sequence
MKFTSVTLACFFFVESSGVSAFTFSTPTQRQRSIPLPIVSSSRRTRSALH

SEQ ID NO: 109 - protein, 50 aa, artificial sequence
  plastid targeting sequence
MKSLVPLAFVLATSSGFAPREHHYYRPNQPARTNALVLQAESTAAVLASA

SEQ ID NO: 110 - protein, 50 aa, artificial sequence
  plastid targeting sequence
MALRRSISRLAMVYLVTLCLKTAFVSAFSSTNTPTTPTSSVQQRSANQQA

SEQ ID NO: 111 - protein, 50 aa, artificial sequence
  plastid targeting sequence
MKFSSIALATVFFAAKAQGFTGSSIKPRFGVQAHSVLKMSTATETDKMAA

SEQ ID NO: 112 - protein, 50 aa, artificial sequence
  plastid targeting sequence
MMMKRALVVLTLSVGVSARASAFAPGAAVKNHAGATQSAIHKQTPFPTTE

SEQ ID NO: 113 - protein, 50 aa, artificial sequence
  plastid targeting sequence
MKLHRKGRYRLLVTAVLLGTVCSFVPENLRSGSVRIPRKNANAGSVPGTH

SEQ ID NO: 114 - protein, 50 aa, artificial sequence
  plastid targeting sequence
MKYSILGNILLSLGLVLETTKAWSVPPPPQTSPAPRESSGDSGTSTPVSP

SEQ ID NO: 115 - protein, 50 aa, artificial sequence
  plastid targeting sequence
MGRGVIIFCVKNFAVWLLIITSAVSIQAWIPLPLSATVKARIDSTTLFFS

SEQ ID NO: 116 - protein, 50 aa, artificial sequence
  plastid targeting sequence
MAISRSRRRSNGLGIVLVWTIFISAVWGWTPPLRGSSRLFLDSADPNVWN

SEQ ID NO: 117 - protein, 50 aa, artificial sequence
  plastid targeting sequence
MKLVSRSFCTLAWTCGAAQAWSVAPRTVARNRPAWVAARHPHTPTACAFS

SEQ ID NO: 118 - protein, 50 aa, artificial sequence
  plastid targeting sequence
MRWTCAFLWCVVVPTLHAWVPSTTNPASRIGTRRWEALGDRELEEPRMNP

SEQ ID NO: 119 - protein, 50 aa, artificial sequence
  plastid targeting sequence
MMREQRMLAILWGAGLWFGGSGVHAWQFPNLFTVPIQPSQKFSQGSTAKS

SEQ ID NO: 120 - protein, 50 aa, artificial sequence
  plastid targeting sequence
MKFSAATFAALVGSAAAYSSSSFTGSALKSSASNDASMSMATGMGVNGFG

SEQ ID NO: 121 - protein, 50 aa, artificial sequence
  plastid targeting sequence
MRFAWVVAAGVVLTTTTQALVPLDCTGMGETRTSGIRPIRGLESNMARYA

SEQ ID NO: 122 - protein, 50 aa, artificial sequence
  plastid targeting sequence
MRFSLQSSLAVLLVLQASHAAAFSAPVSSSNGKNGIRSFAPLSMSLDKYA

SEQ ID NO: 123 - protein, 50 aa, artificial sequence
  plastid targeting sequence
MKFAATILALIGSAAAFAPAQTSRASTSLQYAKEDLVGAIPPVGFFDPLG

SEQ ID NO: 124 - protein, 50 aa, artificial sequence
  plastid targeting sequence
MKTAVIASLIAGAAAFAPAKNAARTSVATNMAFEDELGAQPPLGFFDPLG

SEQ ID NO: 125 - protein, 50 aa, artificial sequence
  plastid targeting sequence
MKGYLFATWACLTISSNASTEAFAHRGPRAPCGLHASKLKTMDSGKLVDV

SEQ ID NO: 126 - protein, 50 aa, artificial sequence
  plastid targeting sequence
MKFLGVTSLLCLWSVVNRENVSEAFAPRHQSLSRPSSRTTSAFSRAPILS

SEQ ID NO: 127 - protein, 50 aa, artificial sequence
  plastid targeting sequence
MKFTAACSIALAASASAFAPIPSVSRTTDLSMSLQKDLANVGKVAAAGAL

SEQ ID NO: 128 - protein, 50 aa, artificial sequence
  plastid targeting sequence
MKSIIFASLLTSAAAFAPASSSTTRTATPTALNEEFCRGYVGGESVEPMF

SEQ ID NO: 129 - protein, 50 aa, artificial sequence
  plastid targeting sequence
MKFAILASMLSAAAAFAPASQGAGKASVALNAEKSPAMPFLPYPENLKGY

SEQ ID NO: 130 - protein, 50 aa, artificial sequence
  plastid targeting sequence
MMRSTILAALLASAAAFAPASMQSQRAGSVSLNAEEMSKSIPFLVKPDKL

SEQ ID NO: 131 - protein, 50 aa, artificial sequence
  plastid targeting sequence
MLTLLILMTRLSLSESFGVTTPRIFRPAPCRHTHRPLKTTLRHSTLPSET

SEQ ID NO: 132 - protein, 50 aa, artificial sequence
  plastid targeting sequence
MKYAVFASLLASAAAFAPAAKPAASTSALNAEMSKSMPFLTAPKNTGGYV

SEQ ID NO: 133 - protein, 50 aa, artificial sequence
  plastid targeting sequence
MKLSLAILALCASTNAAFAPSVSQRTPRDLAGVVAPTGFFDPAGFAARAD

SEQ ID NO: 134 - protein, 50 aa, artificial sequence
  plastid targeting sequence
MKFAVFAFLLASAAAFAPAQQSARTSVATNMAFENEIGAQQPLGYWDPLG

SEQ ID NO: 135 - protein, 50 aa, artificial sequence
  plastid targeting sequence
MKVATTLTLAFICCASAFGLNGQTTSVMKKVGFDAGSKPMVQAIDVQGNR

SEQ ID NO: 136 - protein, 50 aa, artificial sequence
  plastid targeting sequence
MFILKSPALWLLLYPVVAFTAARANSIRPAAALSVFDLSSVEAVPSRKTK

SEQ ID NO: 137 - protein, 50 aa, artificial sequence
  plastid targeting sequence
MKLVPAWTMMTRNAIFSARNPSHFLEVTALFCILIASGRGARTNTAFVNP

SEQ ID NO: 138 - protein, 50 aa, artificial sequence
  plastid targeting sequence
MKFTGVALAVSLAQQQALSNTGGGIVGAYTVSSPSFFTPKSFGSSFVRGP

SEQ ID NO: 139 - protein, 50 aa, artificial sequence
  plastid targeting sequence
MKLFGVSVVIVVVATAVSLSDSVAAFAMAHGGSHKLSNTALRVTGFEDEL

SEQ ID NO: 140 - protein, 50 aa, artificial sequence
  plastid targeting sequence
MVSSSKSTWMMAGACLILMAFQVQSFTFVPATRATSTVKRVAPAFMSAVA